(19) United States
(12) Patent Application Publication
(10) Pub. No.: US 2020/0222186 A1
(43) Pub. Date: Jul. 16, 2020

(54) TRANSCATHETER METHODS FOR HEART VALVE REPAIR

(71) Applicant: NeoChord, Inc., St. Louis Park, MN (US)

(72) Inventors: Daryl Edmiston, Draper, UT (US); Tyler Nordmann, St. Louis Park, MN (US); Scott LaPointe, St. Louis Park, MN (US); Annette Doxon, St. Louis Park, MN (US); David Blaeser, St. Louis Park, MN (US)

(21) Appl. No.: 16/745,074

(22) Filed: Jan. 16, 2020

Related U.S. Application Data
(60) Provisional application No. 62/792,947, filed on Jan. 16, 2019.

(51) Int. Cl. A61F 2/24; A61B 17/04

(52) U.S. Cl. CPC A61F 2/2457; A61B 17/0469; A61F 2/2466; A61B 2017/0496; A61B 2017/0412; A61B 2017/0464; A61F 2220/0016

(57) ABSTRACT

Disclosed herein are minimally invasive systems and methods for intravascularly accessing the heart and performing a transcatheter repair of a heart valve by inserting one or more sutures as artificial chordae into a heart valve leaflet.

### RELATED APPLICATION

This application claims the benefit of U.S. Provisional Application No. 62/792,947 filed Jan. 16, 2019, which is hereby fully incorporated herein by reference.

### TECHNICAL FIELD

The present invention relates to minimally invasive delivery of a suture into the heart. More particularly, the present invention relates to inserting and anchoring one or more sutures as artificial chordae tendineae for a flailing or prolapsing leaflet in a beating heart.

### BACKGROUND

The mitral and tricuspid valves inside the human heart include an orifice (annulus), two (for the mitral) or three (for the tricuspid) leaflets and a subvalvular apparatus. The subvalvular apparatus includes multiple chordae tendineae, which connect the mobile valve leaflets to muscular structures (papillary muscles) inside the ventricles. Rupture or elongation of the chordae tendineae results in partial or generalized leaflet prolapse, which causes mitral (or tricuspid) valve regurgitation. A commonly used technique to surgically correct mitral valve regurgitation is the implantation of artificial chordae (usually 4-0 or 5-0 Gore-Tex sutures) between the prolapsing segment of the valve and the papillary muscle.

This technique for implantation of artificial chordae was traditionally done by an open heart operation generally carried out through a median sternotomy and requiring cardiopulmonary bypass with aortic cross-clamp and cardioplegic arrest of the heart. Using such open heart techniques, the large opening provided by a median sternotomy or right thoracotomy enables the surgeon to see the mitral valve directly through the left atriotomy, and to position his or her hands within the thoracic cavity in close proximity to the exterior of the heart for manipulation of surgical instruments, removal of excised tissue, and/or introduction of an artificial chordae through the atriotomy for attachment within the heart. However, these invasive open heart procedures in which the heart is stopped beating produce a high degree of trauma, a significant risk of complications, an extended hospital stay, and a painful recovery period for the patient. Moreover, while heart valve surgery produces beneficial results for many patients, numerous others who might benefit from such surgery are unable or unwilling to undergo the trauma and risks of such open heart techniques.

Techniques for minimally invasive thoracoscopic repair of heart valves while the heart is still beating have also been developed. U.S. Pat. No. 8,465,500 to Speziali, which is incorporated by reference herein, discloses a thoracoscopic heart valve repair method and apparatus. Instead of requiring open heart surgery on a stopped heart, the thoracoscopic heart valve repair methods and apparatus taught by Speziali utilize fiber optic technology in conjunction with transesophageal echocardiography (TEE) as a visualization technique during a minimally invasive surgical procedure that can be utilized on a beating heart. More recent versions of these techniques are disclosed in U.S. Pat. Nos. 8,758,393 and 9,192,374 to Zentgraf, which are also incorporated by reference herein and disclose an integrated device that can enter the heart chamber, navigate to the leaflet, capture the leaflet, confirm proper capture, and deliver a suture as part of a mitral valve regurgitation (MR) repair. In some procedures, these minimally invasive repairs are generally performed through a small, between the ribs access point followed by a puncture into the ventricle through the apex of the heart. Although far less invasive and risky for the patient than an open heart procedure, these procedures still require significant recovery time and pain.

Some systems have therefore been proposed that utilize a catheter routed through the patient's vasculature to enter the heart and attach a suture to a heart valve leaflet as an artificial chordae. While generally less invasive than the approaches discussed above, transcatheter heart valve repair can provide additional challenges. For example, with all artificial chordae replacement procedures, in addition to inserting a suture through a leaflet, the suture must also be anchored at a second location, such as at a papillary muscle in the heart, with a suture length, tension and positioning of the suture that enables the valve to function naturally. If the suture is too short and/or has too much tension, the valve leaflets may not properly close. Conversely, if the suture is too long and/or does not have enough tension, the valve leaflets may still be subject to prolapse. Proper and secure anchoring of the suture away from the leaflet is therefore a critical aspect of any heart valve repair procedure for inserting an artificial chordae. In the case of transcatheter procedures, such anchoring can be difficult because it can be difficult for the flexible catheter required for routing through the patient's vasculature to apply sufficient force to stably insert traditional suture anchors into the heart wall, e.g., the myocardium.

### SUMMARY

Disclosed herein are minimally invasive systems and methods for intravascularly accessing the heart and performing a transcatheter repair of a heart valve by inserting one or more sutures as artificial chordae into a heart valve leaflet.

In an embodiment, a method of repairing a heart valve includes intravascularly accessing an interior of the heart and inserting one or more sutures into a heart valve leaflet of the heart. The one or more sutures can be attached to a suture anchor exterior to the heart and the suture anchor advanced into the heart anchored into a heart wall of the heart with an anchor delivery catheter. A tension of the one or more sutures can then be adjusted to achieve proper heart valve function. Once the desired tension has been achieved, a suture lock on the suture anchor can be actuated to retain the one or more sutures at the suture anchor at the tension that achieves proper heart valve function.

In an embodiment, a method of repairing a heart valve includes initially inserting one or sutures into a heart valve leaflet with a leaflet capture catheter. The free ends of the sutures can then be threaded through an anchor externally of the body and the anchor advanced into the heart with an anchor catheter. The anchor is implanted into the heart wall with the anchor catheter and the tension of the sutures can be adjusted for proper valve function. Once an appropriate tension is achieved, the anchor can be actuated to lock the sutures in place with respect to the anchor. The free ends of the sutures can then be crimped and cut to leave the anchor and sutures in place to repair valve function.

In an embodiment, a method of repairing a heart valve utilizes a two-piece anchor and includes first implanting an anchor body into the heart wall with an anchor catheter. The anchor can include a guidewire extending out of the body to enable access to the anchor body. One or more sutures can then be inserted into a heart valve leaflet with a leaflet capture catheter. The free ends of the sutures can be interfaced with an anchor lock external of the body and the anchor lock and sutures advanced into the heart and to the anchor body with the guidewire. The anchor lock can be initially attached to the anchor body in an unlocked position to enable the sutures to be tensioned and then actuated into a locked position on the anchor body once proper tension has been set. The free ends of the sutures can then be crimped and cut to leave the anchor and sutures in place to repair valve function.

In an embodiment, a method of repairing a heart valve uses a modular anchor and includes first implanting an anchor body attached to a guidewire into the heart wall with an anchor catheter. One or more sutures can then be inserted into a heart valve leaflet with a leaflet capture catheter. Individual anchor tabs can be interfaced with the free ends of each suture external to the body. Each anchor tab can be individually and sequentially attached to a guide rail that slides along the guidewire to guide the anchor tabs to the anchor body. Each suture can be individually tensioned through the anchor tab attached to the anchor body. Once each of the anchor tabs has been delivered to the anchor body and each of the sutures has been tensioned, an anchor cap can lock the sutures with respect to the anchor body. The free ends of the sutures can then be crimped and cut to leave the anchor and sutures in place to repair valve function.

In an embodiment, a method of repairing a heart valve includes initially interfacing an anchor suture with an anchor external to the body and then inserting the anchor into the heart wall with a suture loop and a suture free end of the anchor suture remaining external to the body. A leaflet capture catheter carrying a leaflet suture can then be inserted through the suture loop of the anchor suture and into the body to insert one or more leaflet sutures into the leaflet. After inserting the leaflet sutures, the free ends of the leaflet sutures will be within the anchor suture loop and pulling on the free end of the anchor suture from outside of the body will cause the suture loop of the anchor to tighten around the free ends of the leaflet sutures and draw them down onto the anchor. The leaflet sutures can then be tensioned, and the anchor suture cut and crimped to lock the leaflet sutures on the anchor. The free ends of the leaflet sutures can then be crimped and cut to leave the anchor and sutures in place to repair valve function.

In an embodiment, a system for repairing a heart valve in a beating heart of a patient includes an elongate flexible guide catheter configured to be inserted into the heart through the vasculature of the patient to provide a pathway into the heart from outside the body and an elongate flexible anchor catheter configured to be inserted into the heart through the elongate flexible guide catheter. The system further includes a suture anchor configured to interface with a suture and be anchored in a heart wall of the heart with the anchor catheter to enable the suture to function as an artificial chordae extending between the anchor and a heart valve leaflet in the heart. The system also includes a suture lock configured to selectively lock the suture on the suture anchor under tension.

Various embodiments of systems, devices, and methods have been described herein. These embodiments are given only by way of example and are not intended to limit the scope of the present invention. It should be appreciated, moreover, that the various features of the embodiments that have been described may be combined in various ways to produce numerous additional embodiments. Moreover, while various materials, dimensions, shapes, implantation locations, etc. have been described for use with disclosed embodiments, others besides those disclosed may be utilized without exceeding the scope of the invention.

### BRIEF DESCRIPTION OF THE DRAWINGS

Subject matter hereof may be more completely understood in consideration of the following detailed description of various embodiments in connection with the accompanying figures, in which:

FIG. 1 is a schematic representation of a method for inserting a leaflet capture catheter into a beating heart of a patient according to an embodiment.

FIGS. 2A-2H depict schematic representations of various steps of a method of repairing a heart valve according to an embodiment.

FIGS. 3A-3M depict schematic representations of various steps of a method of repairing a heart valve according to an embodiment.

FIGS. 4A-4K depict schematic representations of various steps of a method of repairing a heart valve according to an embodiment.

FIGS. 5A-5L depict schematic representations of various steps of a method of repairing a heart valve according to an embodiment.

FIGS. 6A-6C depict schematic representations of various steps of a method of repairing a heart valve according to an embodiment.

FIGS. 7A-7D depict schematic representations of various steps of a method of repairing a heart valve according to an embodiment.

FIGS. 8A-8J depict schematic representations of various steps of a method of repairing a heart valve according to an embodiment.

FIG. 9 depicts a flowchart of steps in a method of repairing a heart valve according to an embodiment.

FIG. 10 depicts a flowchart of steps in a method of repairing a heart valve according to an embodiment.

FIG. 11 depicts a flowchart of steps in a method of repairing a heart valve according to an embodiment.

FIGS. 12A-121 depict various views of an anchor assembly that can be used with the methods as disclosed herein and FIGS. 13A-13H depict the various components thereof.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The present disclosure is generally directed to inserting and anchoring one or more sutures as artificial chordae into one or more heart valve leaflets through an intravascular, transcatheter approach. A heart valve leaflet may be captured and a suture inserted through the leaflet in any manner known in the art. Examples of such leaflet capture catheters are disclosed in copending U.S. Patent Publication No. 2019/0290260 and U.S. patent application Ser. No. 16/564,887, each of which is hereby incorporated by reference herein. Another transcatheter procedure for inserting an artificial chordae is disclosed in U.S. Patent Publication No. 2016/0143737, which is hereby incorporated by reference herein.

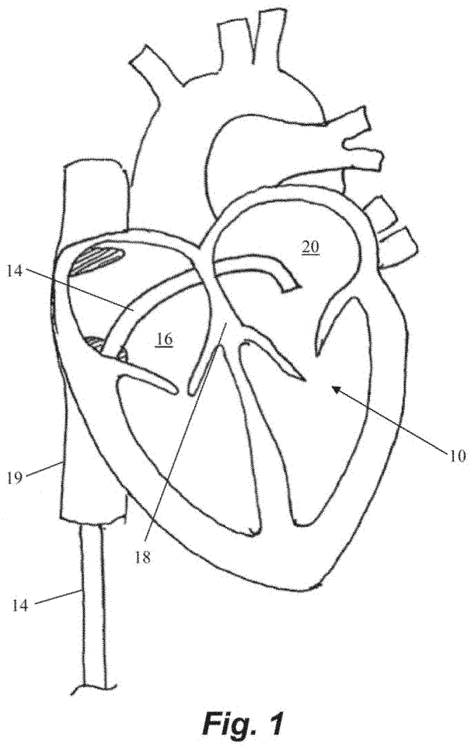

In each of the below described embodiments, access into the heart to the valve being repaired can be gained through an intravascular, transcatheter approach. If the valve being repaired is the mitral valve, the valve may further be accessed transseptally. FIG. 1 depicts a schematic representation of an embodiment of an access approach for a heart valve repair system accessing the mitral valve 10. FIG. 1 depicts an elongate flexible guide catheter 14 accessing the interior of the heart via the femoral vein. In some embodiments, such a system can further include an outer guide catheter and an inner guide catheter. In such embodiments, the outer guide catheter can be inserted into the femoral vein at the patient's groin and advanced through the femoral vein into the inferior vena cava 19 and then into the right atrium 16. In various embodiments, the outer guide catheter can be steerable in a single plane and can have an outer diameter of about or less than about 30 french, such as, for example 24 french. The septum 18 can then be punctured using an appropriate puncture tool and the outer guide catheter advanced into the septum 18 or through the septum 18 into the left atrium 20. The inner guide catheter can then be axially advanced through the outer guide catheter into the left atrium 20. In some embodiments, the inner guide catheter can have two plans of steerability and can be maneuvered along with and/or beyond the outer guide catheter to establish a stable position superior to the mitral valve 10 and to provide a desired trajectory for operation of a leaflet capture catheter to repair the valve.

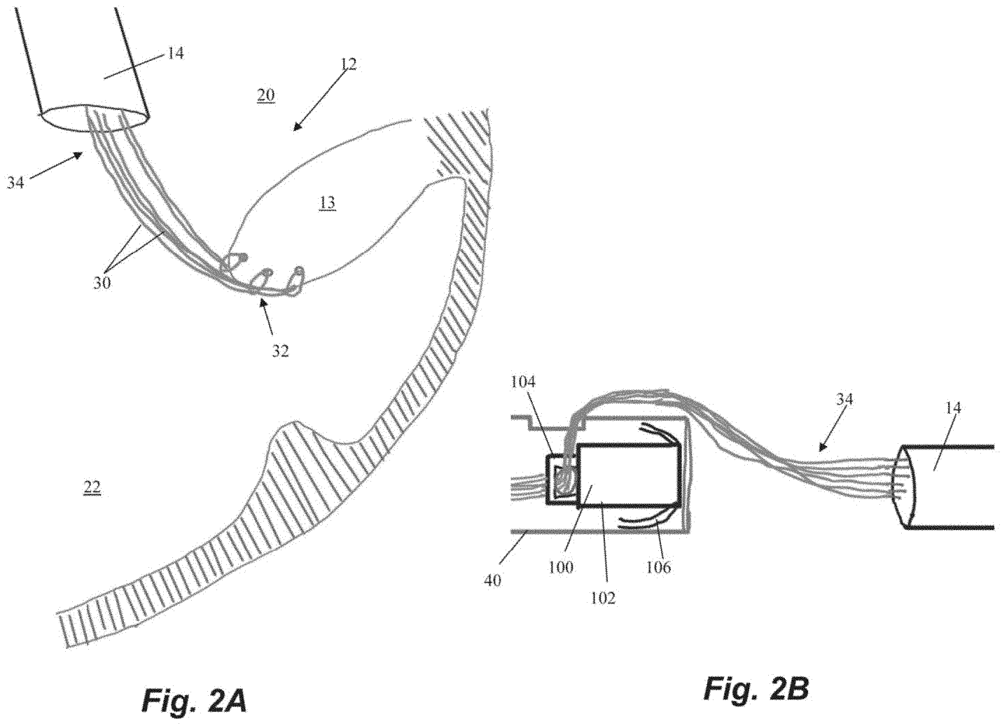

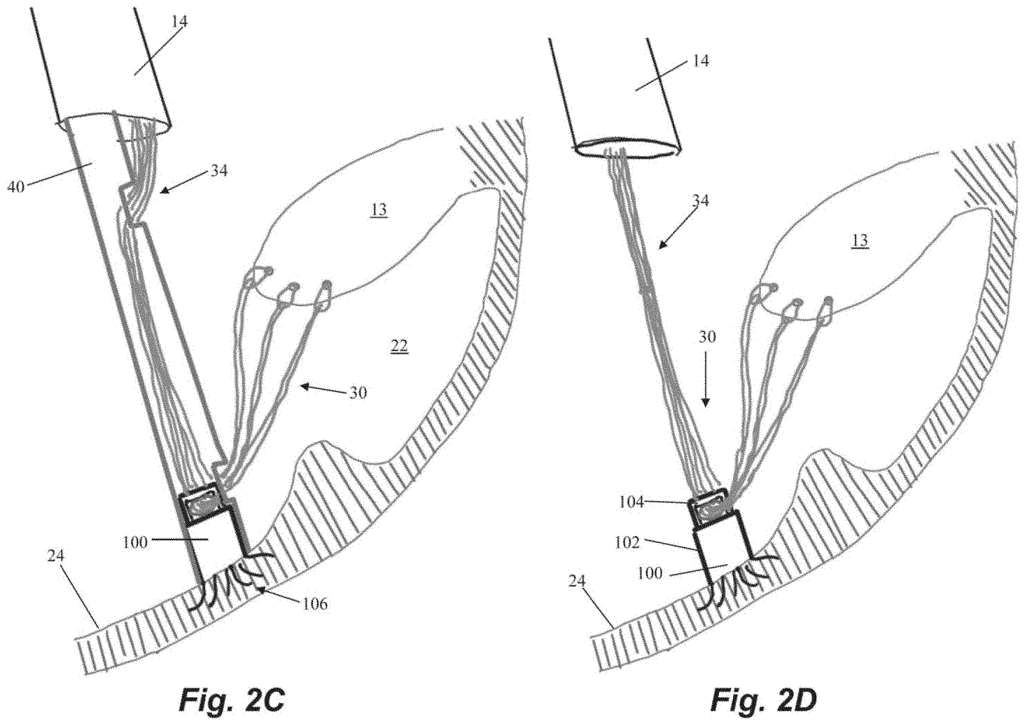

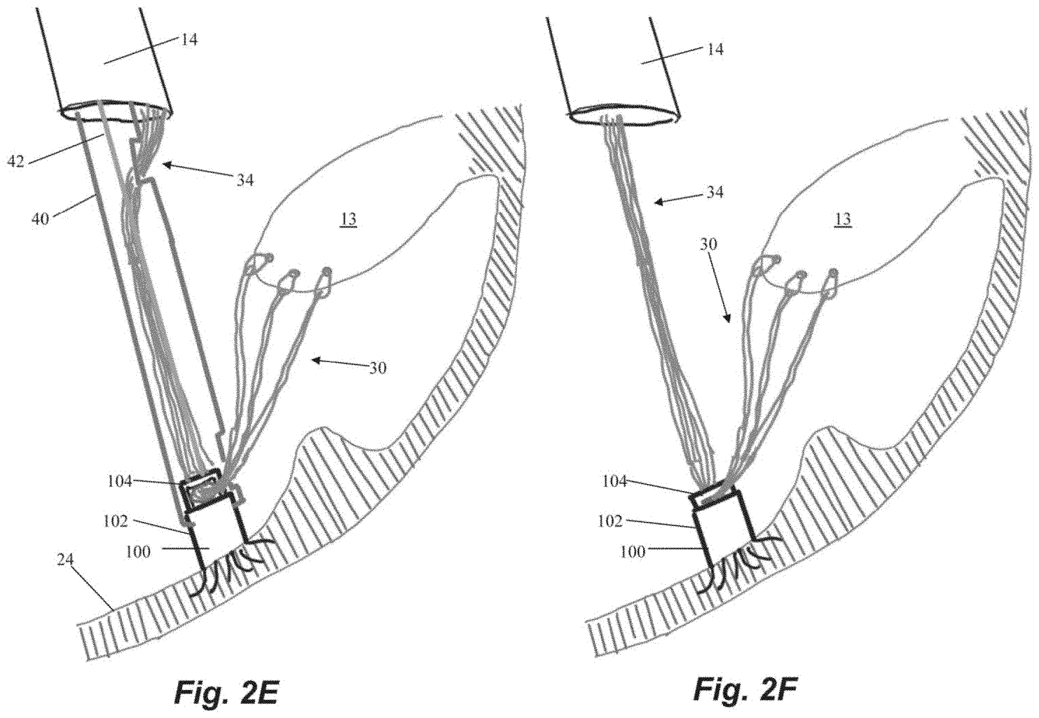

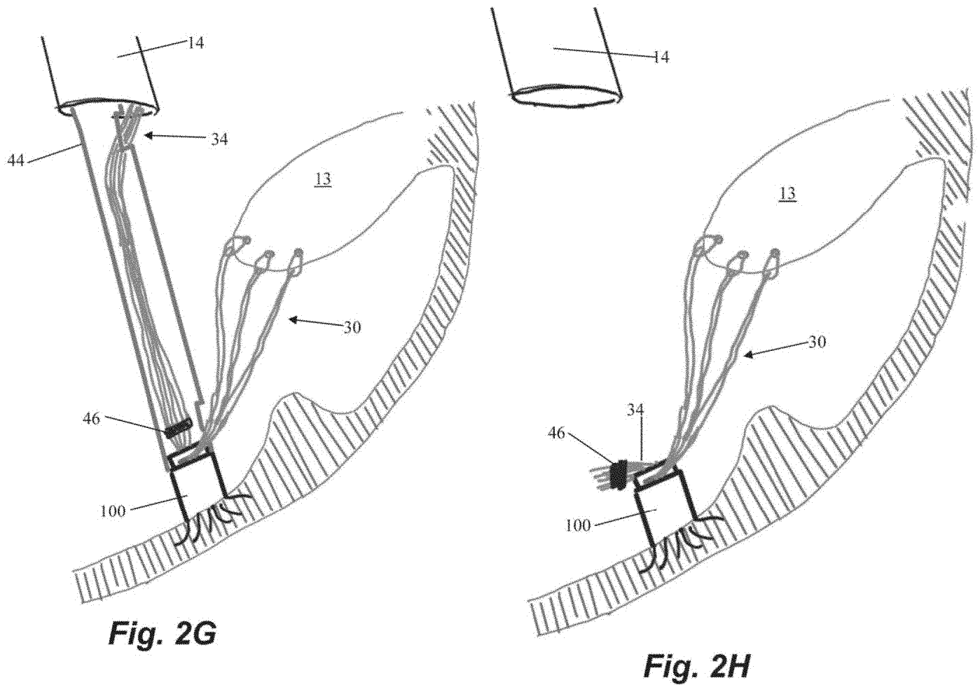

Schematic representations of various steps of an embodiment of a method of repairing a heart valve are depicted in FIGS. 2A-2H. Initially, one or more sutures 30 are inserted through a leaflet 13 in the valve 12 as depicted in FIG. 2A. In the depicted embodiment, three sutures 30 have been inserted, but greater or fewer sutures can be inserted. The sutures 30 can be inserted with a leaflet capture catheter, as described above, to each form a girth hitch knot 32 around the edge of the leaflet 13 and, at the stage of the procedure depicted in FIG. 2A, have a pair of free ends 34 extending back through the guide catheter 14 and out of the body.

The free ends 34 of the sutures 30 are then fed through an anchor 100 external to the body. Anchor 100 can include an anchor body 102 and a locking head 104 and sutures 30 can be passed through an opening between anchor body 102 and locking head 104 as depicted in FIG. 2B. Anchor 100 can further include a plurality of tissue engaging features configured to retain the anchor 100 in the heart, such as a plurality of tines 106 extending from anchor body 100. Anchor 100 can be disposed in an anchoring catheter 40 for delivery into the body through the guide catheter 14. In one embodiment, tissue-engaging features such as tines 106 are comprised of shape memory material that is constrained by the anchor catheter 40. As the anchor 100 is advanced out of the anchor catheter 40 and into the heart wall 24, as depicted in FIG. 2C, the tines 106 embed themselves in the trabeculae tissue of the heart wall 24 to retain the anchor 40 therein.

Once the anchor 100 is deployed into the heart wall 24, the anchor catheter 40 is withdrawn, as depicted in FIG. 2D, and the sutures 30 can be tensioned for proper valve function as is known in the art. Suture 30 tension can be adjusted by moving free ends 34 from outside of the heart with respect to the opening between the anchor body 102 and the anchor head 104 through which the free ends extend. After the sutures 30 have been tensioned, the anchor 100 can be locked to hold the sutures 30 at that tension. Referring to FIG. 2E, the anchor catheter 40 can be reintroduced into the heart through the guide catheter 14 with a lock actuator 42 extending through the anchor catheter 40 to push the anchor head 104 down towards the anchor body 102 and lock the sutures 30 in place therebetween as shown in FIG. 2F. In some embodiments, locking actuator 42 can be a flexible wire configured to actuate the anchor head 104. In various embodiments, the locking actuator 42 can actuate the anchor head with, e.g., a compressive force, rotational force, etc.

After the sutures 30 are locked in place with respect to the anchor 100, the free ends 34 of the suture 30 can be severed. Referring to FIGS. 2G-2H, a cutting catheter 44 can be advanced into the heart through the guide catheter 14 and to the anchor 100 to cut the free ends 34 of the suture 30 adjacent to the anchor 100 with a cutting element (not depicted). Cutting catheter 44 can also be employed to advance a crimping element 46 along the free ends to adjacent the anchor 30 to hold the severed free ends 34 together. Crimping element 46 can be disposed on the distal end of cutting catheter 44 to enable the crimping element 46 to be advanced to the anchor 100 and then detached from the cutting catheter 44 by, for example, a twisting motion. The cutting element would then be employed to cut the free ends 34 of the suture 30 after the crimping element 46 has been secured. The heart valve 12 has now been repaired and the system can be withdrawn from the heart and the surgical access sealed.

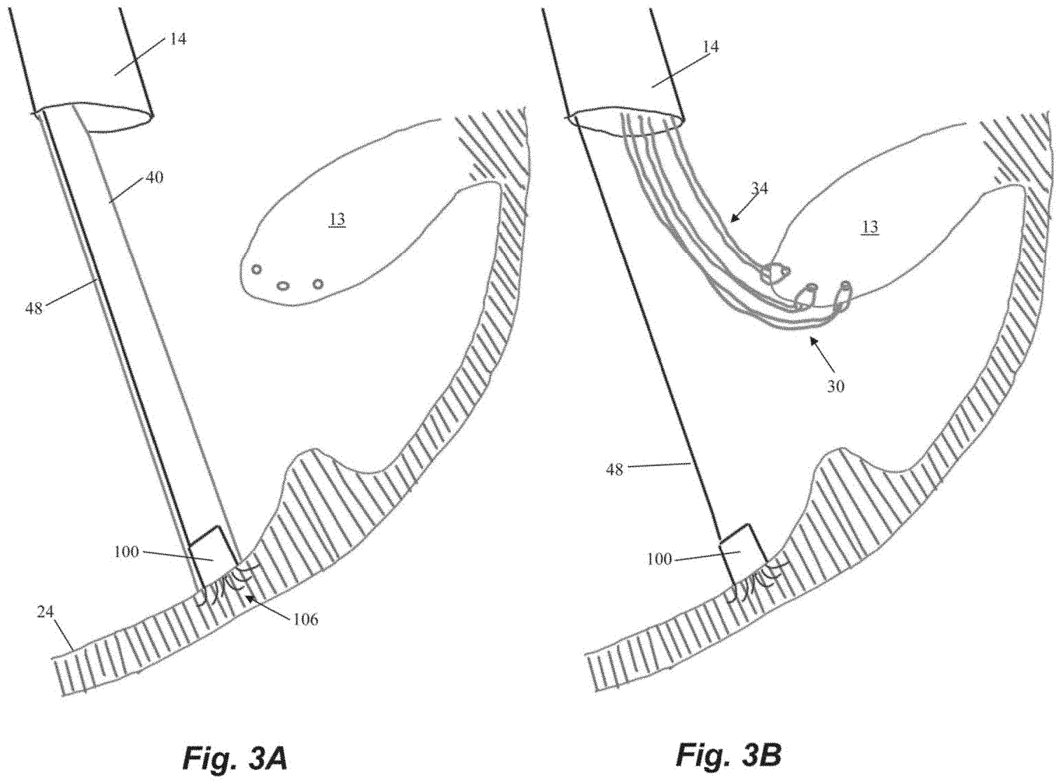

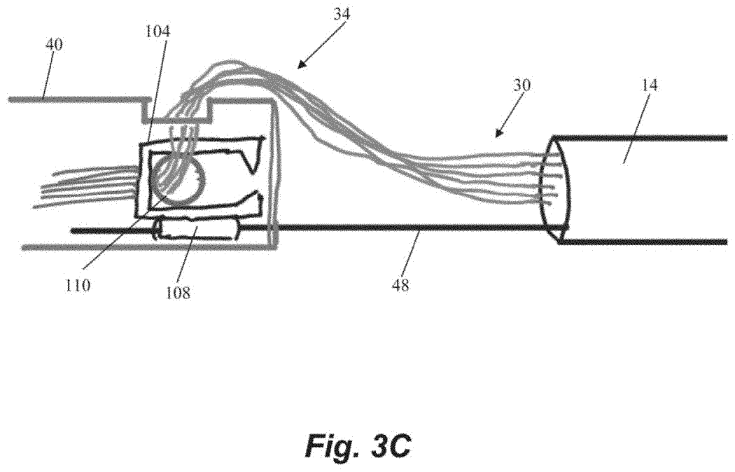

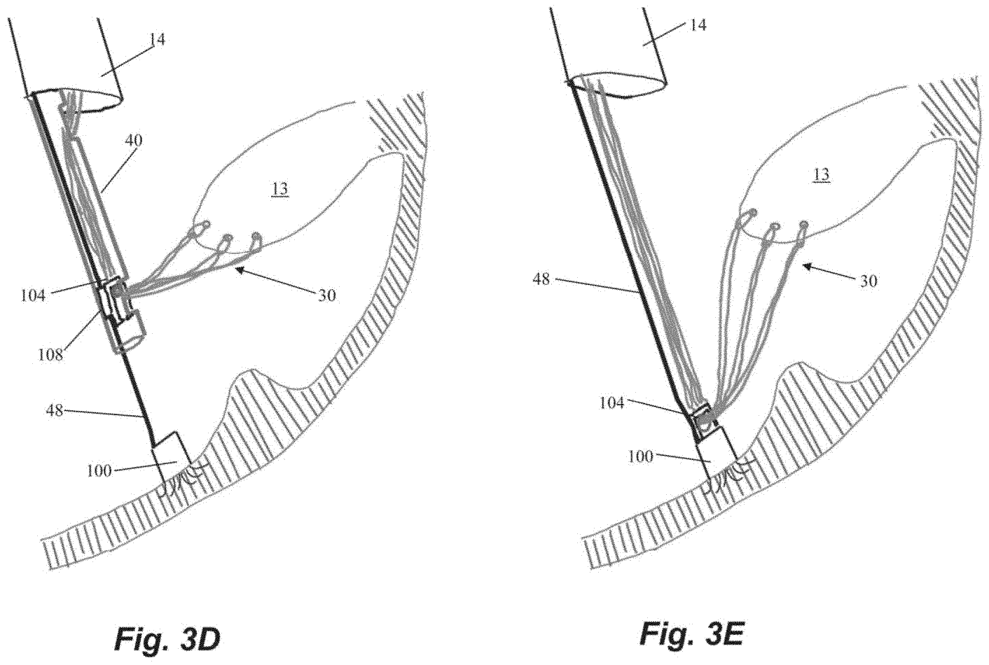

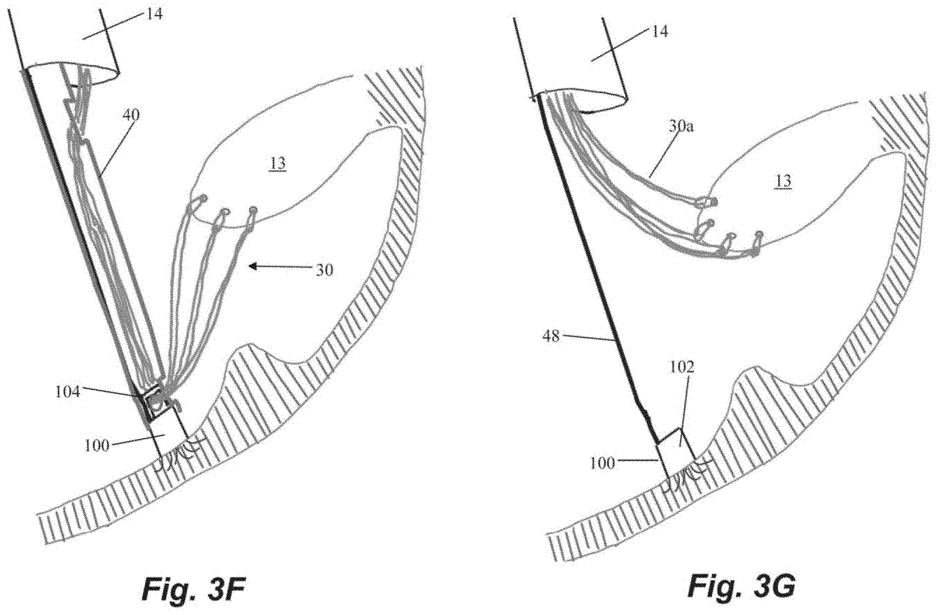

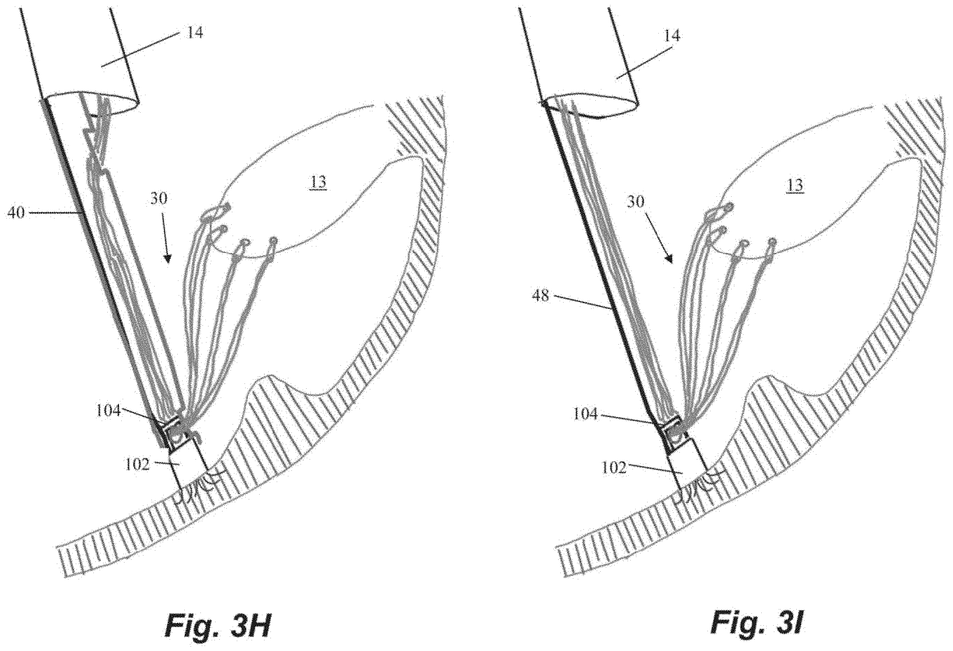

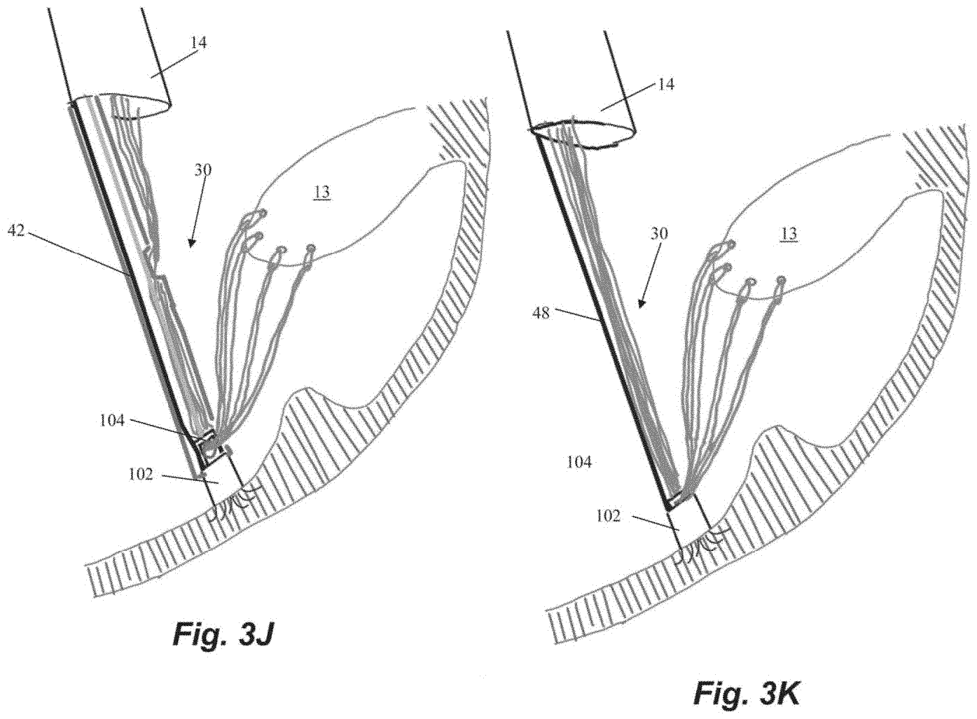

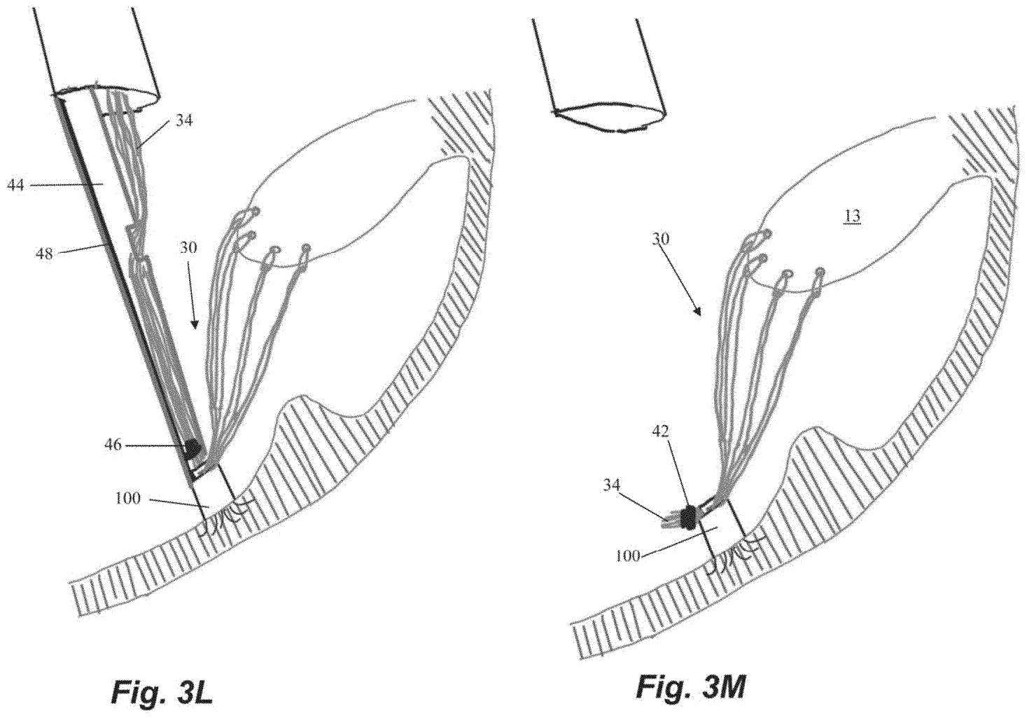

FIGS. 3A-3M depict schematic representations of various steps of another embodiment of a method of repairing a heart valve. In this embodiment, an anchor 100 is first seated in the heart wall 24 with an anchor catheter 40 as described above. In contrast to the embodiment of FIGS. 2A-2H, anchor 100 is initially implanted as just an anchor body 102 with a plurality of tines 106 or other anchoring feature (no locking head 104) as depicted in FIGS. 3A-3B. As there are no sutures attached to anchor 30 when it is implanted in this embodiment, a guidewire 48 as shown in FIGS. 3A-3B can extend from anchor to the exterior of the body to enable access to the anchor body 102 from outside of the body. Following implantation of the anchor, the anchor catheter 40 is withdrawn and one or more sutures 30 can be inserted into the leaflet 13 with a leaflet capture catheter as described above.

The free ends 34 of the sutures 30 can then be threaded from outside the body through an aperture 110 in a separate anchor locking head 104 disposed in anchor catheter 40 as shown in FIG. 3C. Locking head 104 can include a guide flange 108 configured to guide the locking head 104 along guidewire 48. The locking head 104 is then advanced to the anchor body 102 along the guidewire 48 as depicted in FIG. 3D. Referring to FIG. 3E, the locking head 104 can initially be loosely positioned on the anchor 100 to enable the sutures 30 to be tensioned as described above.

One advantage of the embodiment described in FIGS. 3A-3M with a two-piece anchor is enabling use of additional sutures if it is determined during tensioning that additional sutures are desired. Referring to FIGS. 3F-31, in a situation where the surgeon determines that more sutures should be implanted, the anchor catheter 40 can be advanced back down to the anchor 100 to engage and remove the locking head 104 as depicted in FIG. 3F. One or more additional sutures 30a can then be inserted through leaflet 13 with the leaflet capture catheter (FIG. 3G). The one or more additional sutures 30a can also be inserted through aperture 110 in locking head 104 as previously depicted in FIG. 3C and the steps of engaging the locking head 104 with the anchor body 102 and tensioning the sutures 30 repeated as depicted in FIGS. 3H-31.

Once the desired tension has been achieved, the locking head 104 can be locked on the anchor body 102 with locking actuator 42 as described above and shown in FIGS. 3J-3K. The cutting catheter 44 can them be employed to crimp the sutures ends 34 together with the crimping element 46 as described above and can sever both the suture ends 34 and the guidewire 48 with the cutting element to complete the repair process.

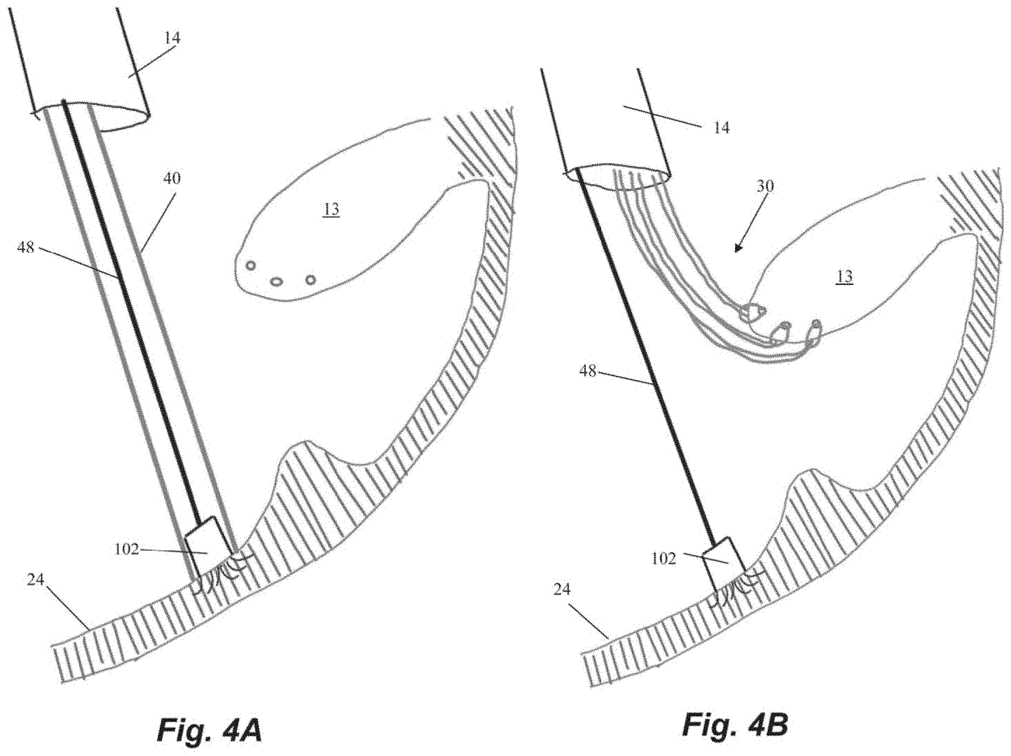

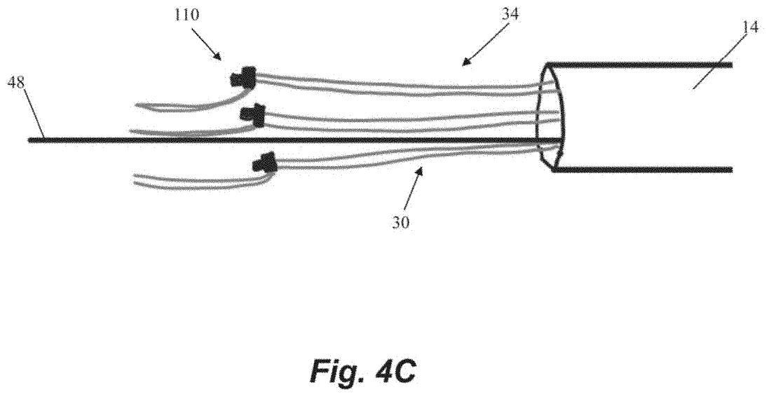

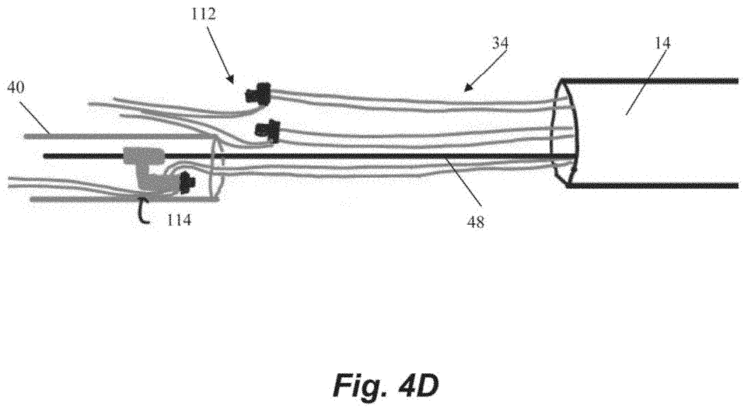

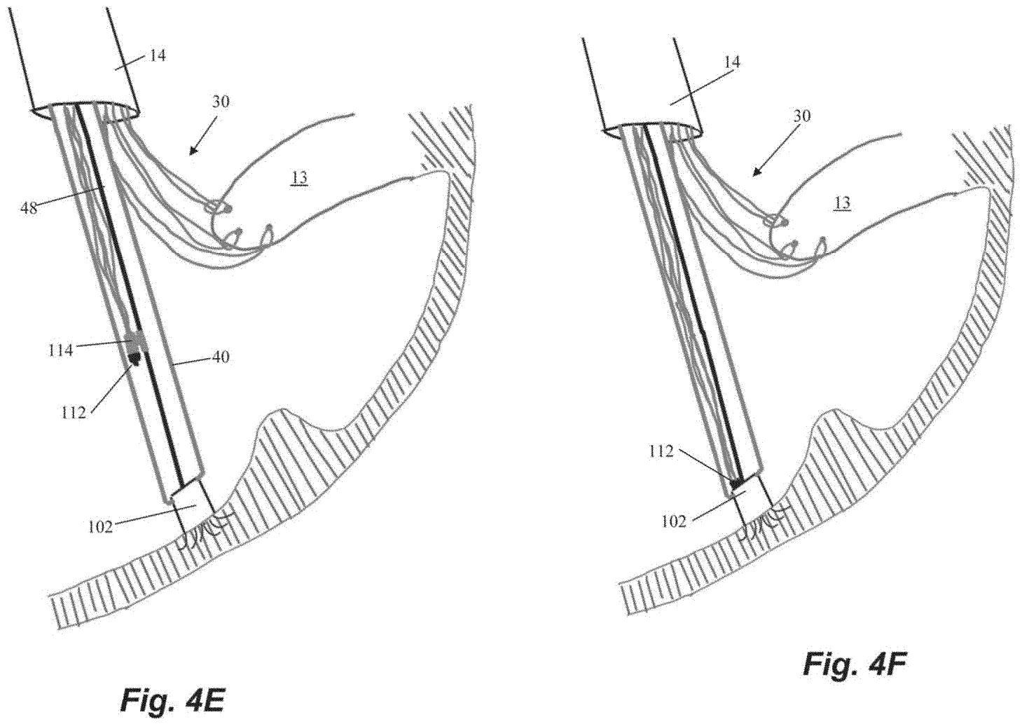

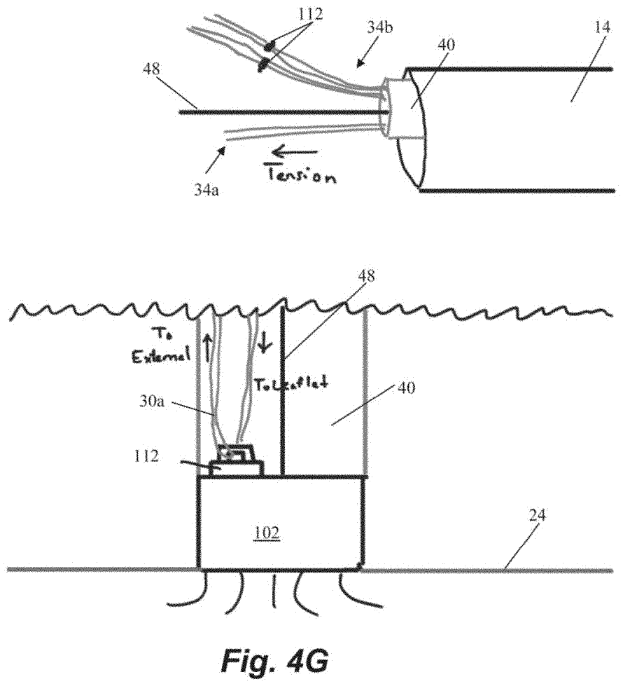

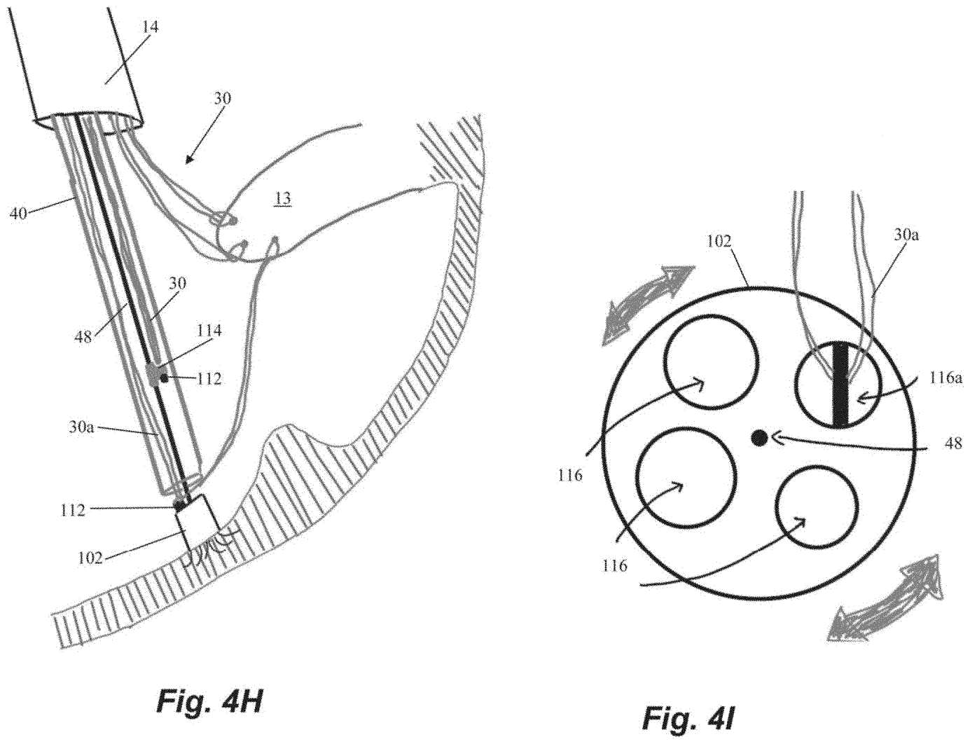

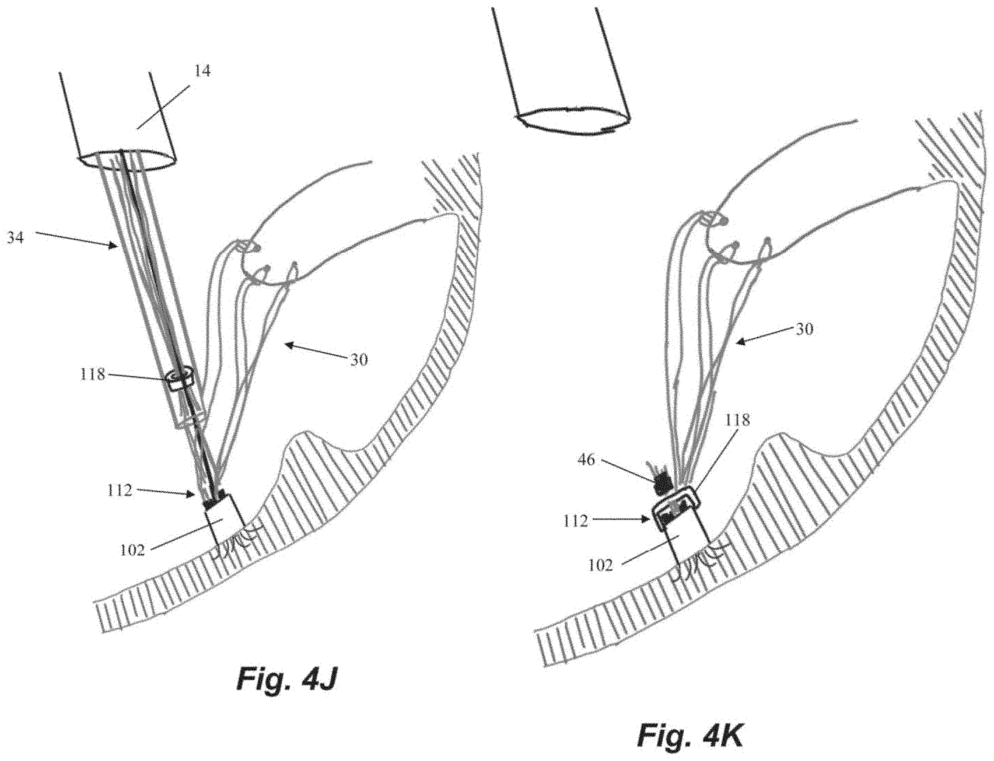

Another embodiment of steps for repairing a heart valve is schematically depicted in FIGS. 4A-4K. As shown in FIGS. 4A-4B, the method begins similarly to the previous method in that an anchor body 102 with an attached guidewire 48 is first implanted into the heart wall 24 and then one or more sutures 30 are inserted through the leaflet. The suture ends 34 in the depicted method are provided with individual modular anchor tabs 112 rather than a single locking head for all sutures 30 as in the previous embodiments. Each anchor tab 112 can selectively and sequentially interface with a rail guide 114 carried by the anchor catheter 40 that can slide along guidewire 48 as shown in FIG. 4D. FIGS. 4E-4F depict how the rail guide 114 can be advanced along the guide wire 48 to attach the modular anchor tab 112 to the anchor body 102. Referring to FIG. 4G, the suture ends 34a extending from the anchored suture 30a can be used to adjust the tension. The other suture ends 34b have not yet been anchored in this figure.

Referring now to FIGS. 4H-41, another anchor tab 112 can now be attached to the rail guide 114 and the rail guide 114 is rotated about the guidewire 48 in order to advance the anchor tab to a different aperture 116 in the anchor body 102 than the aperture 116a to which the first suture 30a was attached and anchored. Once each of the sutures 30 has been attached to the anchor body 102 with a corresponding anchor tab 112 and adjusted to an appropriate tension, an anchor cap 118 can be advanced along the sutures 30 to the anchor body 102 to lock the sutures 30 with respect to the anchor tabs 112 as shown in FIG. 4J. The suture ends 34 can then be cut and/or crimped as described above.

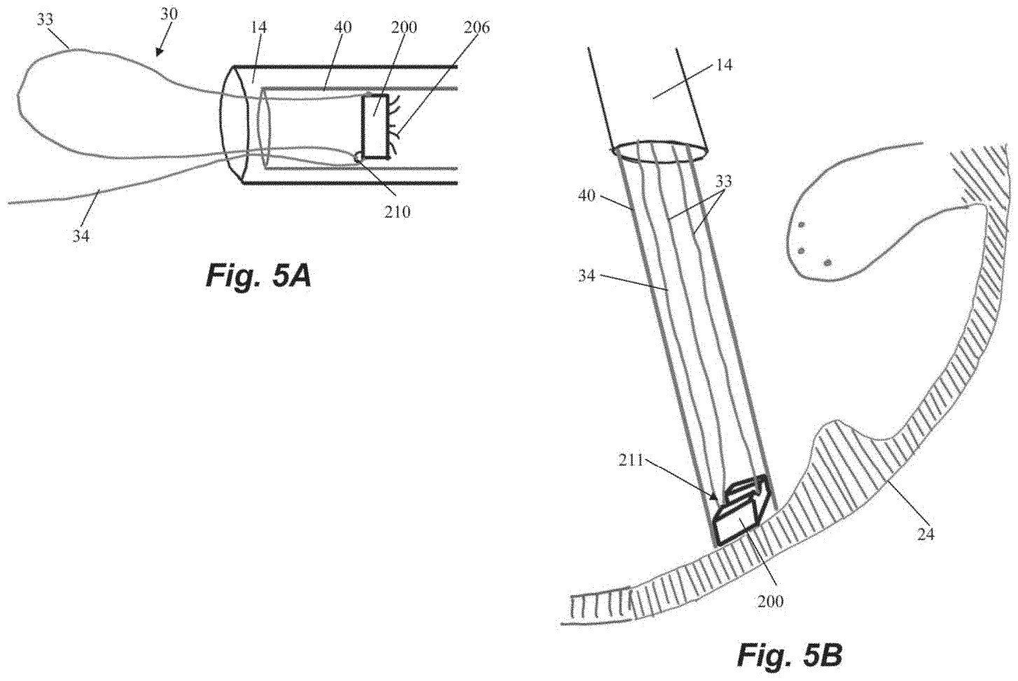

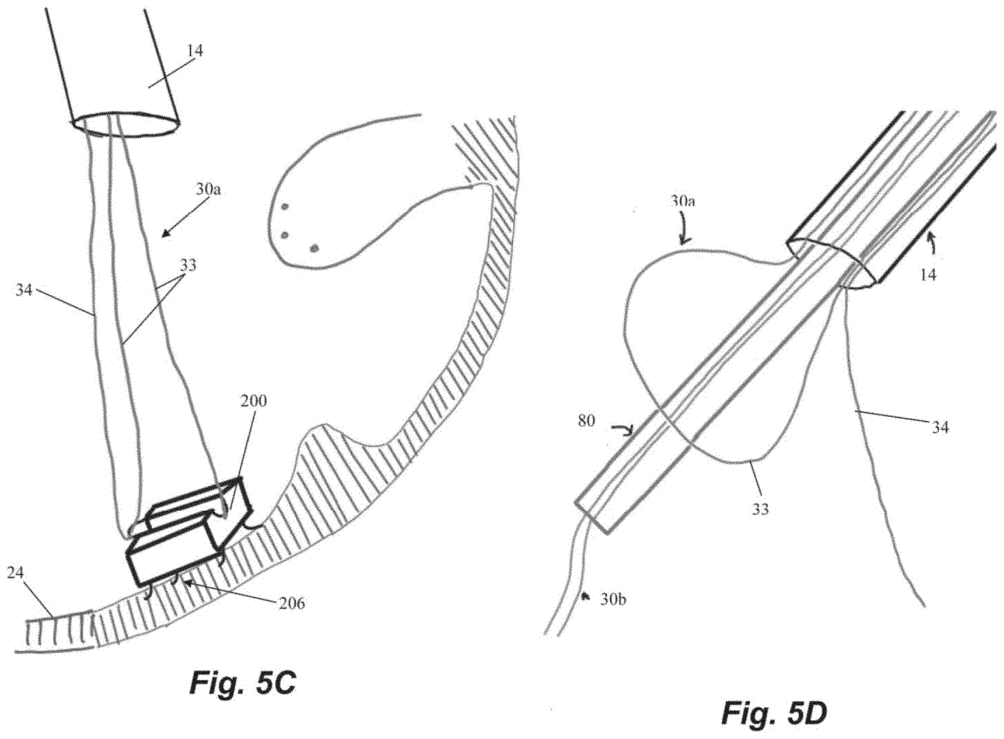

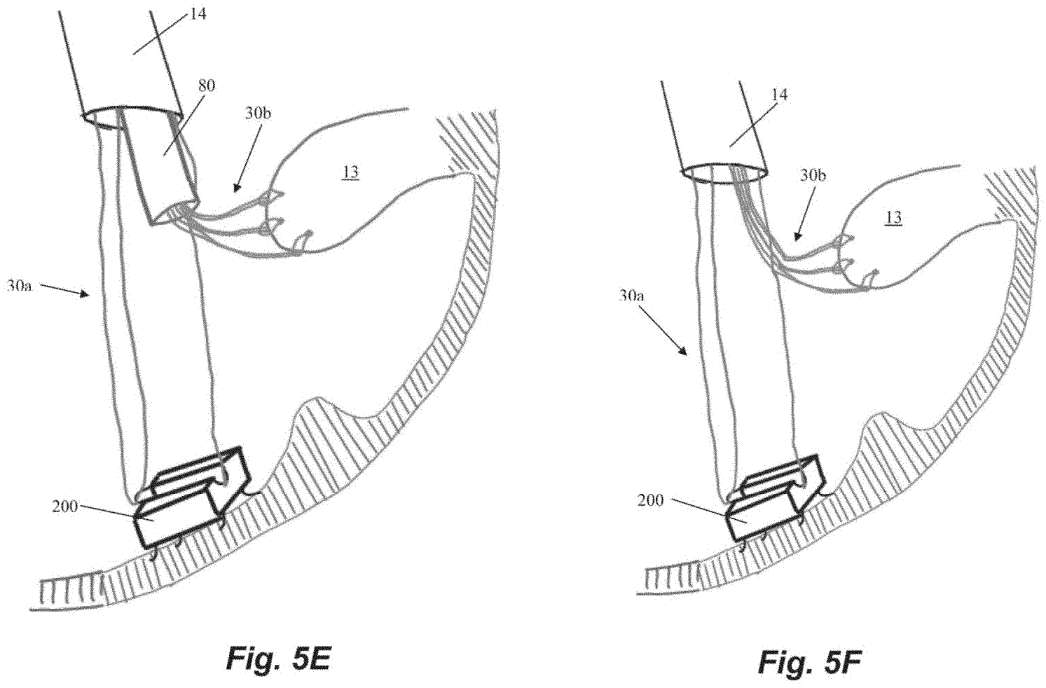

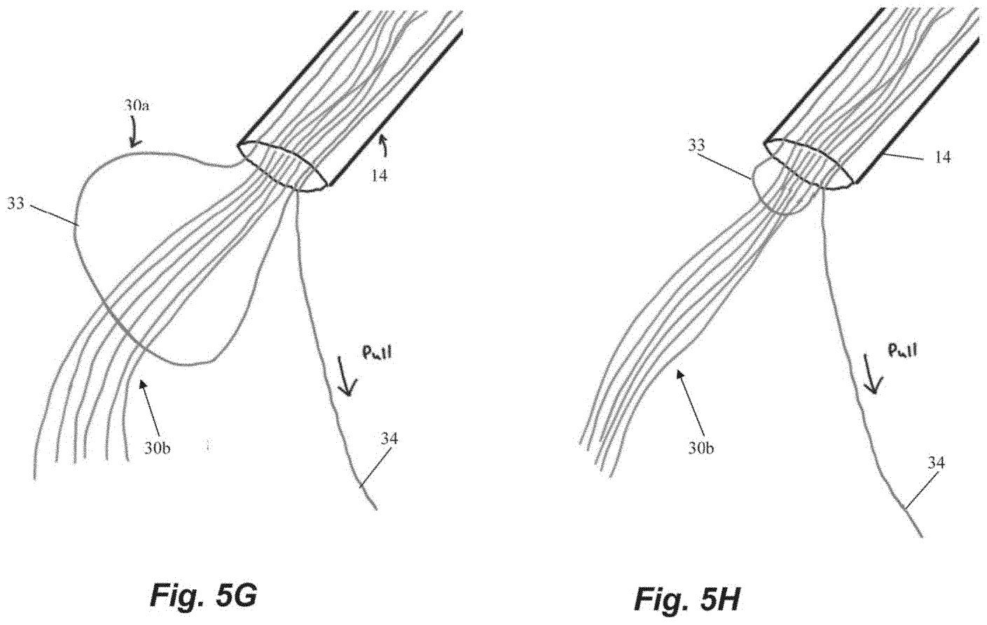

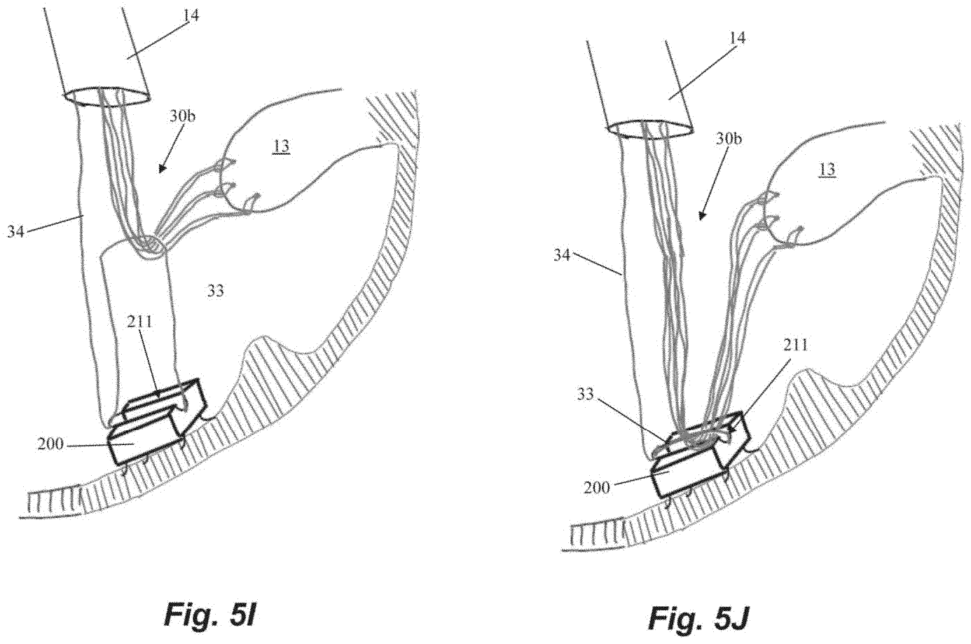

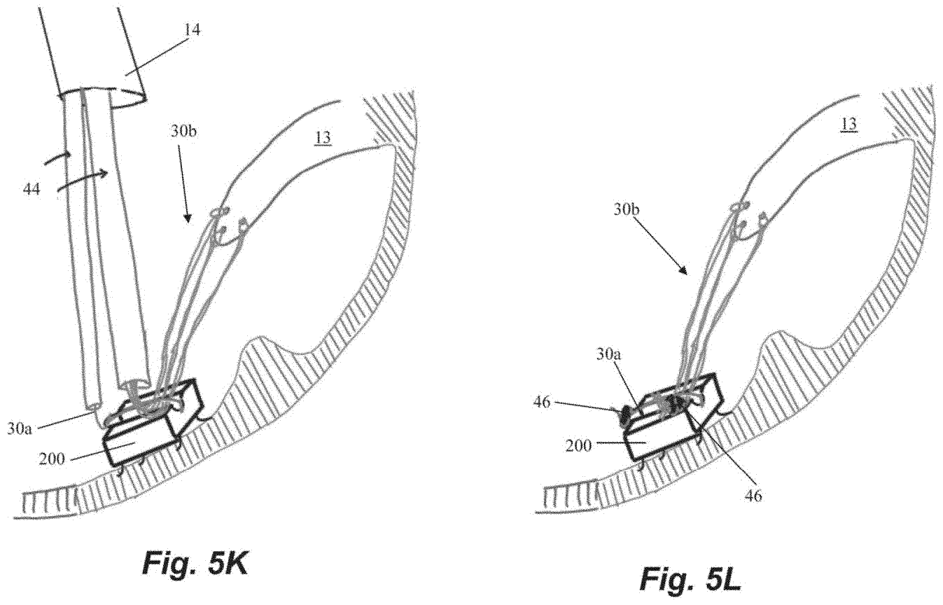

FIGS. 5A-5L depict schematic representations of various steps of a method of repairing a heart valve according to another embodiment. In this embodiment, an anchor suture 30a is first attached to an anchor 200. As shown in FIG. 5A, anchor 200 includes a loop 210 in an anchor channel 211 and suture 30a can be threaded through loop 210 such that a suture loop 33 and a single free end 34 extend from anchor 200. Anchor 200 can further include a plurality of anchor tines 206 or other tissue engaging features. Anchor 200 can then be seated in the heart wall 24 with anchor catheter 40 as described herein. A leaflet capture catheter 80 carrying a chordal suture 30b is inserted through the loop 33 of the anchor suture 30a and into the guide catheter 14 for introduction into the body as shown in FIG. 5D. One our more chordal sutures 30b can be deployed into the leaflet 13 in this manner as described herein and as depicted in FIGS. 5E-5F.

The chordal sutures 30b can be secured to the anchor 200 by pulling on the free end 34 of the anchor suture 30a externally of the body to cause the loop 33 of the anchor suture 30a to pull the chordal sutures 30b down to the anchor 200 and into channel 211 as depicted in FIGS. 5G-5J. The chordal sutures 30b can then be tensioned, cut and crimped and the free end 34 of the anchor suture 30a cut and crimped with one or more cut and crimp catheters 44 as described above.

Although the above figures depict anchors 100, 200 having a plurality of tines that embed into the heart wall to secure the anchor in the heart, it should be understood that such anchors are only one embodiment of the disclosure. Various other anchors can be interchangeably employed in each of the above-described systems. Such anchors can include those disclosed in U.S. Patent Application Publication Nos. 2019/0343626; 2019/0343633 and 2019/0343634, which are hereby incorporated herein by reference. Other anchors that could be employed in the above described system include helical or corkscrew type anchors that are rotated by an anchoring catheter extending outside of the body to secure the anchor to the heart wall. Examples of such anchors are disclosed in U.S. Provisional Patent Application Nos. 62/834,512, which is hereby incorporated by reference.

Although the suture locks 104 described herein for locking the tensioned sutures with respect to the corresponding anchor body 102 have been depicted and described as locking heads that are linearly pushed or pulled to clamp or release the sutures, it should be understood that such suture locking is only one embodiment of the disclosure. Various other methods of releasably holding one or more sutures under tension can be employed. For example, in other embodiments the sutures can be clamped by rotationally engaging the sutures. In such an embodiment, one or more sutures can be threaded through a portion of the anchor such that when a rotationally clamping element is rotated by an anchor catheter, the clamping element tightens on the suture to clamp the suture between the clamping element and another portion of the anchor. In embodiments, the clamping element can also be rotated in the opposite direction to release the suture, enabling retensioning, and, in the case of a selectively attachable clamping element, withdrawal of the clamping element from the anchor body to enable additional sutures to be inserted into the leaflet and subsequently tensioned along with the other sutures.

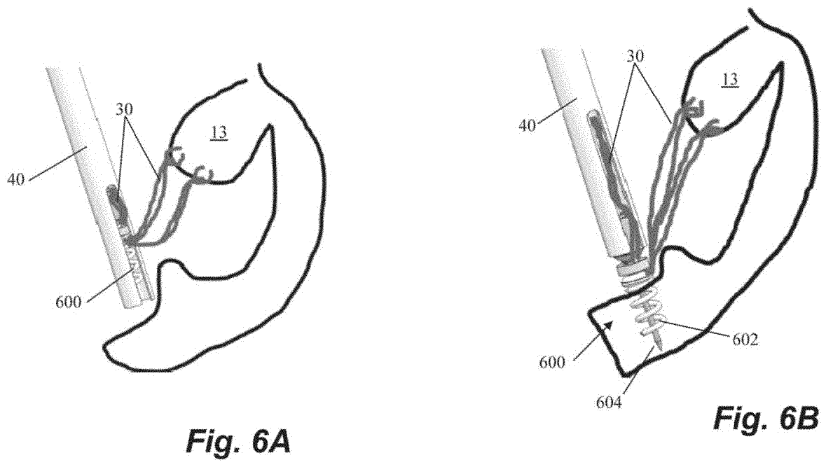

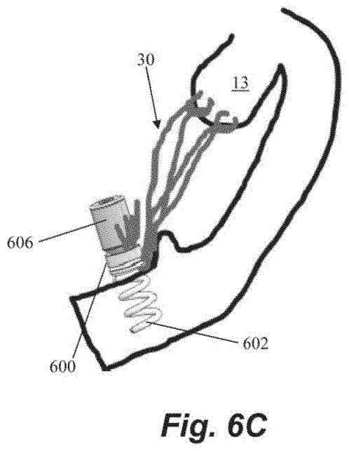

FIGS. 6A-6C depict schematic representations of various steps of a method of repairing a heart valve according to another embodiment that utilizes a helical or corkscrew type anchor 600 that includes an anchor coil 602 and a stabilizing needle 604 extending longitudinally through the anchor coil. In the depicted embodiment, after the sutures 30 are inserted into the leaflet 13, they can be threaded through anchor 600 outside of the body. The anchor 600 can then be inserted into anchor delivery catheter 40 and positioned adjacent the heart wall. The stabilizing needle 604 first pierces the tissue to stabilize the anchor while the coil 602 is driven into the tissue by rotating the anchor 600. In this and other embodiments, stabilizing needle can hold the position of the anchor coil against rotational forces transmitted from the catheter that may cause movement of the coil away from the heart wall. In some embodiments, the coil 602 can be inserted generally perpendicularly to the interior surface of the heart wall. In other embodiments, due to the interior geometry of the hard the coil 602 may be inserted at a non-perpendicular angle to the heart wall. After the anchor 600 has been inserted, the sutures can be tensioned and then locked by rotating an anchor clamp 606 to clamp down on the sutures 30. The stabilizing needle 604 can then be removed and the sutures ends severed as depicted in FIG. 6C.

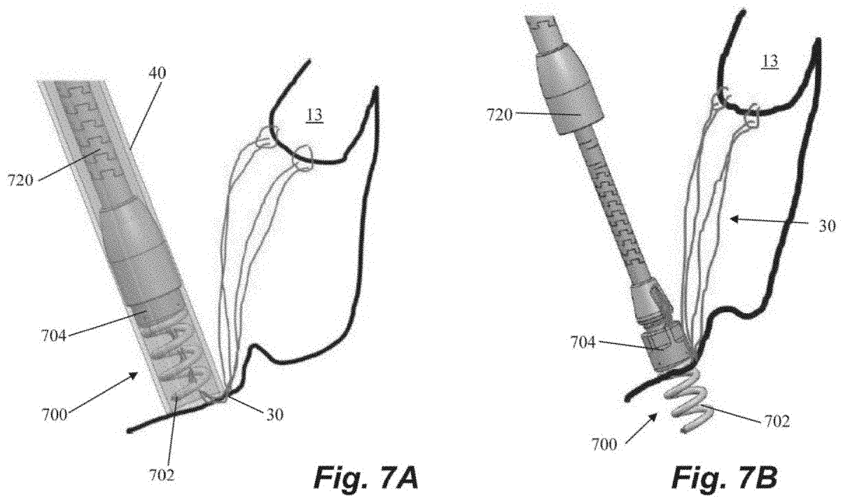

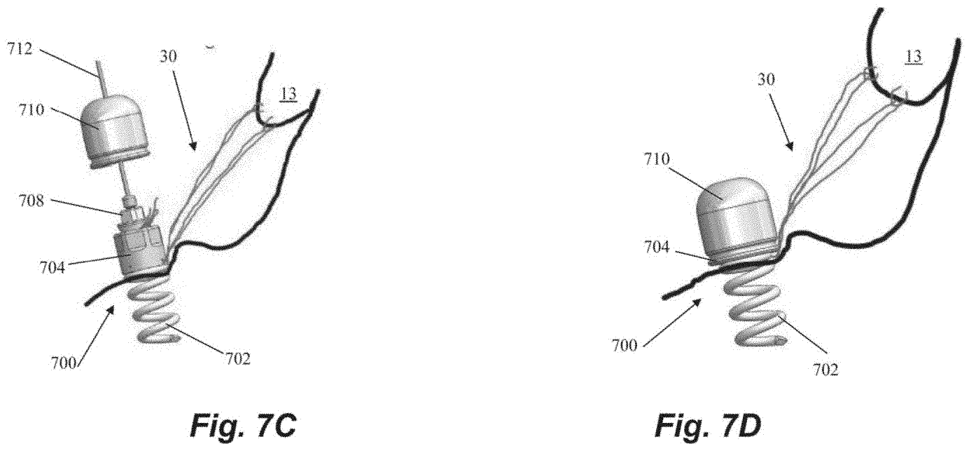

FIGS. 7A-7D depict schematic representations of various steps of a method of repairing a heart valve according to another embodiment that also utilizes a helical or corkscrew type anchor 700 that includes an anchor coil 702. After sutures 30 are inserted into the leaflet 13, the sutures 30 can be threaded through the anchor coil 702 and anchor body 704 of anchor exterior to the body. Anchor catheter 40 can then be used to deliver the anchor 700 and a corresponding anchor driver 720 into the heart. The anchor driver 720 can then be used to rotate the anchor to 700 embed the coil 702 into the heart wall, which causes the suture 30 to slide through the coil and to the anchor body 704, and then the anchor driver 720 can be withdrawn as depicted in FIG. 7B. In some embodiments, the coil 702 can be inserted generally perpendicularly to the interior surface of the heart wall. In other embodiments, due to the interior geometry of the hard the coil 702 may be inserted at a non-perpendicular angle to the heart wall. The sutures can then be tensioned and locked under tension by rotating a suture lock 708 (see FIG. 7C) to clamp the suture in anchor body 704. The anchor driver 720 can then be withdrawn, leaving a tether 712 extending from the anchor 700 back out of the heart. The free ends of the suture 30 can then be severed and a suture cover 710 can be advanced along the tether 712 to be seated on the anchor body 704 to cover sutures 30. The tether 712 can then be severed and withdrawn from the body, leaving the anchor 700 in place.

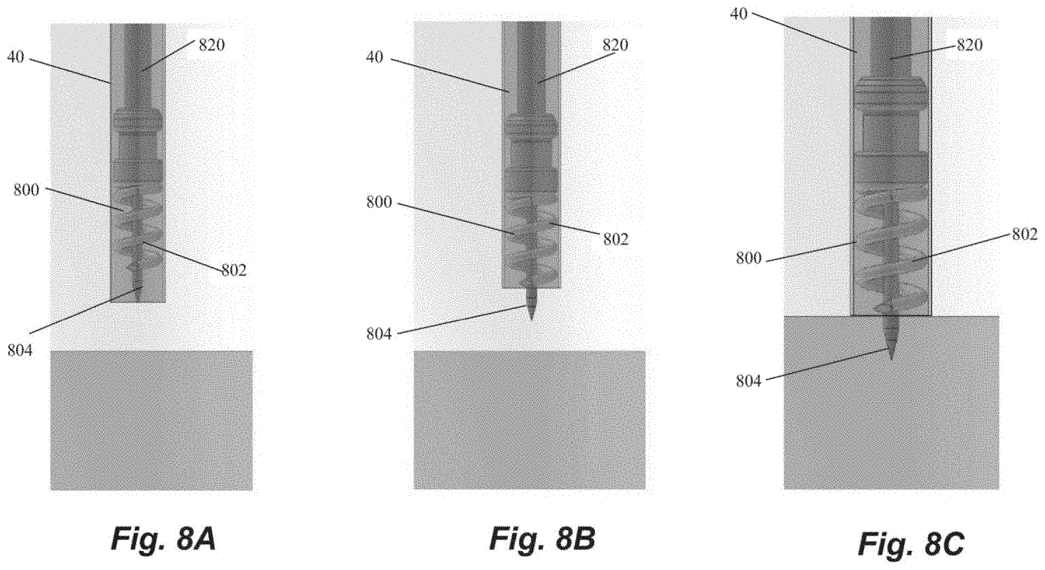

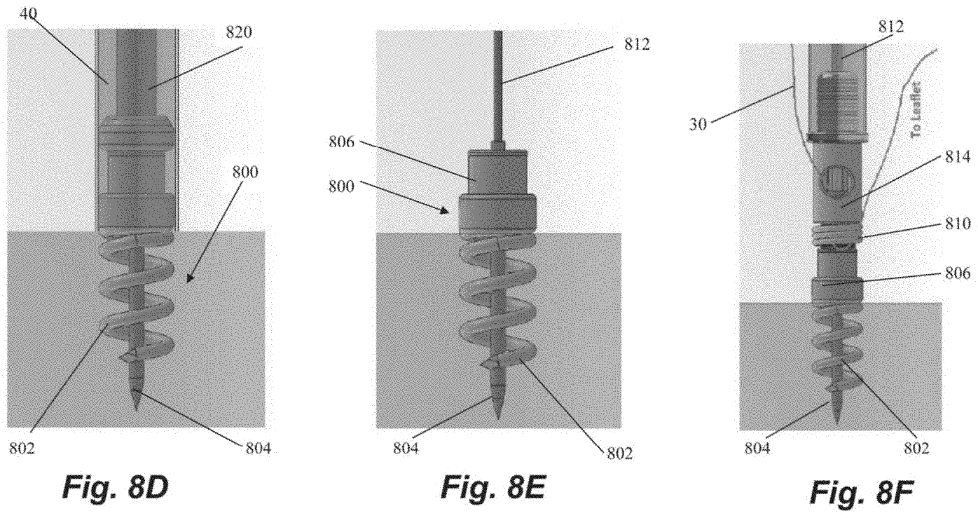

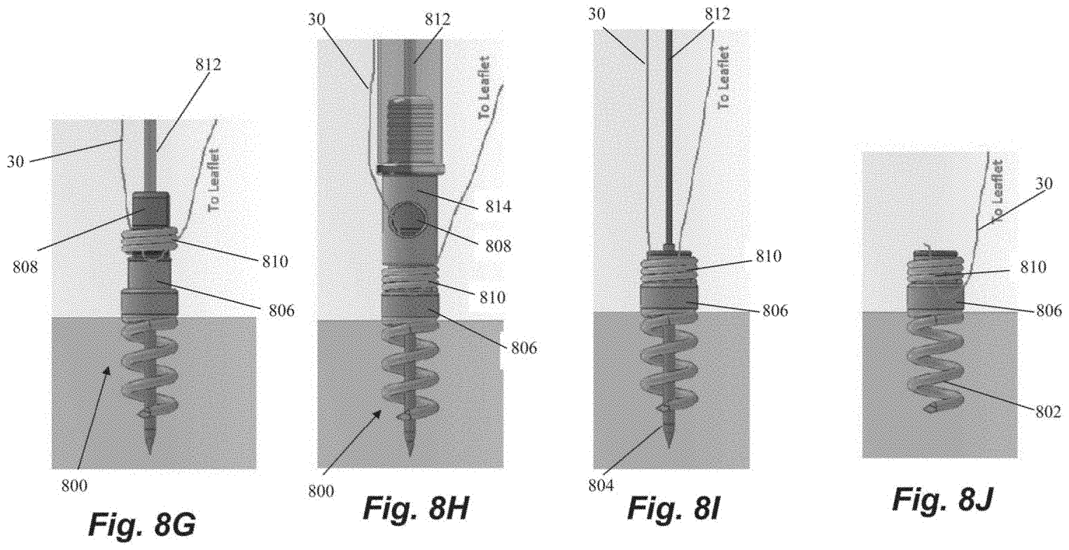

FIGS. 8A-8J depict schematic representations of various steps of a method of repairing a heart valve according to another embodiment that utilizes a helical or corkscrew type anchor 800 that includes an anchor coil 802 and a stabilizing needle 804 extending longitudinally through the anchor coil. An anchor delivery catheter 40 delivers the anchor 800 into the heart and the anchor 800 is partially rotated out of the catheter 40 with an anchor driver 820 to expose the stabilizing needle 804 to enable insertion of the needle 804 into the heart wall. The anchor 800 is then further rotated to insert the anchor coil 802 into the heart tissue and the anchor catheter 40 and anchor driver 820 withdrawn as depicted in FIGS. 8D-8E, leaving a tether 812 in place extending from an anchor hub 806 back out of the heart. In some embodiments, the coil 802 can be inserted generally perpendicularly to the interior surface of the heart wall. In other embodiments, due to the interior geometry of the hard the coil 802 may be inserted at a non-perpendicular angle to the heart wall. A suture lock delivery system can then bring one or more sutures to the anchor 800 along the tether 812 as depicted in FIGS. 8F-8J. The sutures can therefore be inserted into the leaflet either before or after the anchor is seated in the heart wall. A spring carrier 808 that holds a locking spring 810 can be left attached to anchor hub 806 as depicted in FIG. 8F. The sutures 30 can then be appropriately tensioned and then the suture lock delivery system is brought back to the anchor as depicted in FIG. 8H with pusher 814 deploying the locking spring 810 off of the spring carrier 808 and onto the anchor hub 806 to clamp the sutures 30 between the locking spring 810 and the anchor hub 806 at the adjusted tension. The suture lock delivery system, including the pusher 814 and spring carrier 808, can then be removed as well as the tether 812 and stabilizing needle 804 and the sutures 30 cut to complete the procedure.

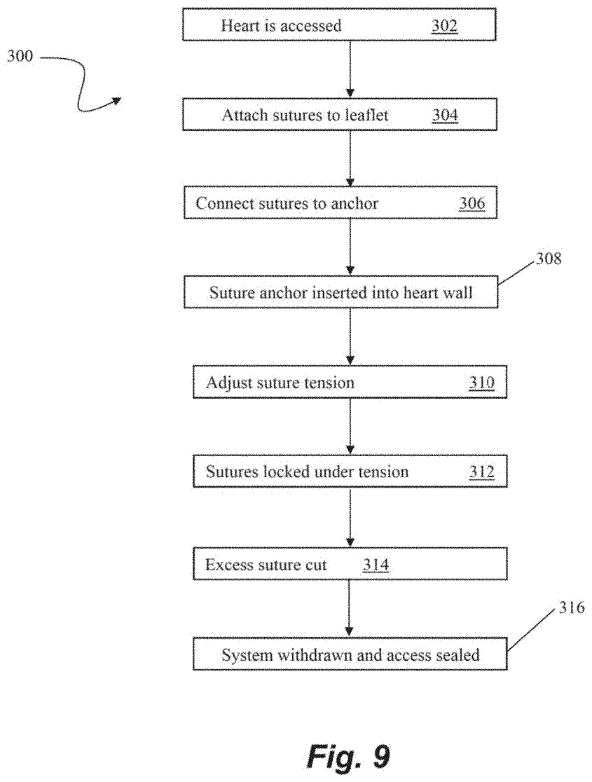

FIG. 9 depicts a flowchart of steps in a method 300 of transcatheter heart valve repair according to an embodiment. At step 302, surgical access to the heart can be gained such as by, for example, the intravascular, transcatheter approach depicted and describe with respect to FIG. 1. One or more sutures can then be attached to a valve leaflet at step 304 with, for example, a leaflet capture catheter such as those previously incorporated by reference herein. The free ends of the sutures can then be threaded through or otherwise connected to a portion of a suture anchor such as those described herein exterior to the heart at step 306. The suture anchor can then be advanced into the heart and embedded into the heart wall at step 308. The tension of the sutures can then be adjusted at step 310 for proper valve function. Once proper tension is achieved, the sutures can be locked at step 312, such as, for example by clamping the sutures with suture lock that is, e.g., pushed or rotated to clamp the sutures against the anchor. In some embodiments, the suture anchor may be capable of being unlocked and the tension readjusted. Any excess suture can then be cut at step 314. The heart valve is now repaired and the system can be withdrawn and the surgical access sealed at step 316.

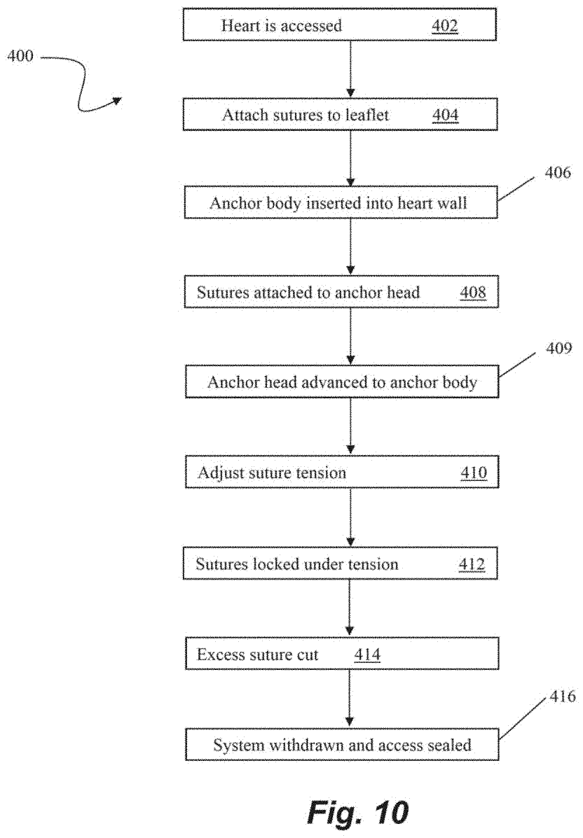

FIG. 10 depicts a flowchart of steps in a method 400 of transcatheter heart valve repair according to an embodiment. At step 402, surgical access to the heart can be gained such as by, for example, the intravascular, transcatheter approach depicted and describe with respect to FIG. 1. One or more sutures can then be attached to a valve leaflet at step 404 with, for example, a leaflet capture catheter such as those previously incorporated by reference herein. In this embodiment, an anchor body of a suture anchor can then be inserted into the heart wall at step 406. A tether or guidewire, for example, can extend from the anchor body out of the body to enable access to the anchor body from outside of the body. The free ends of the sutures can then be threaded through or otherwise connected to a portion of an anchor head or other suture locking component such as those described herein exterior to the heart at step 408. At step 409, the anchor head can be advanced along the tether or guidewire into the body and interfaced with the anchor body. The tension of the sutures can then be adjusted at step 410 for proper valve function and the sutures can be locked at step 412 by, for example, clamping the sutures between the anchor head and the anchor body. The anchor head enables not only the ability to unlock the sutures and readjust the tension but can also be removed from the anchor body so that one or more additional sutures can be inserted into the leaflet, then also threaded through the anchor head and locked with the other sutures between the anchor head and the anchor body. Any excess suture can then be cut at step 414, and the heart valve is now repaired and the system can be withdrawn and the surgical access sealed at step 416.

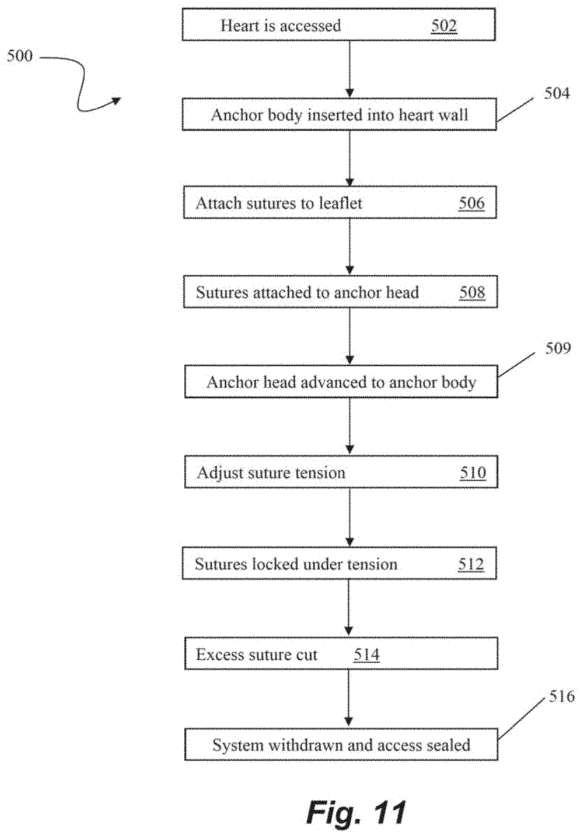

FIG. 11 depicts a flowchart of steps in a method 500 of transcatheter heart valve repair according to an embodiment. At step 502, surgical access to the heart can be gained such as by, for example, the intravascular, transcatheter approach depicted and describe with respect to FIG. 1. In this embodiment, an anchor body of a suture anchor is first inserted into the heart wall at step 504 with, e.g., a tether or guidewire extending from the anchor body out of the body to enable access to the anchor body from outside of the body. One or more sutures can then be attached to a valve leaflet at step 506 with, for example, a leaflet capture catheter such as those previously incorporated by reference herein. The free ends of the sutures can then be threaded through or otherwise connected to a portion an anchor head or other suture locking component such as those described herein exterior to the heart at step 508. At step 509, the anchor head can be advanced along the tether or guidewire into the body and interfaced with the anchor body. The tension of the sutures can then be adjusted at step 510 for proper valve function and the sutures can be locked at step 512 by, for example, clamping the sutures between the anchor head and the anchor body. The anchor head of this embodiment also enables not only the ability to unlock the sutures and readjust the tension, but can also be removed from the anchor body so that one or more additional sutures can be inserted into the leaflet, then also threaded through the anchor head and locked with the other sutures between the anchor head and the anchor body. Any excess suture can then be cut at step 4514, and the heart valve is now repaired and the system can be withdrawn and the surgical access sealed at step 516.

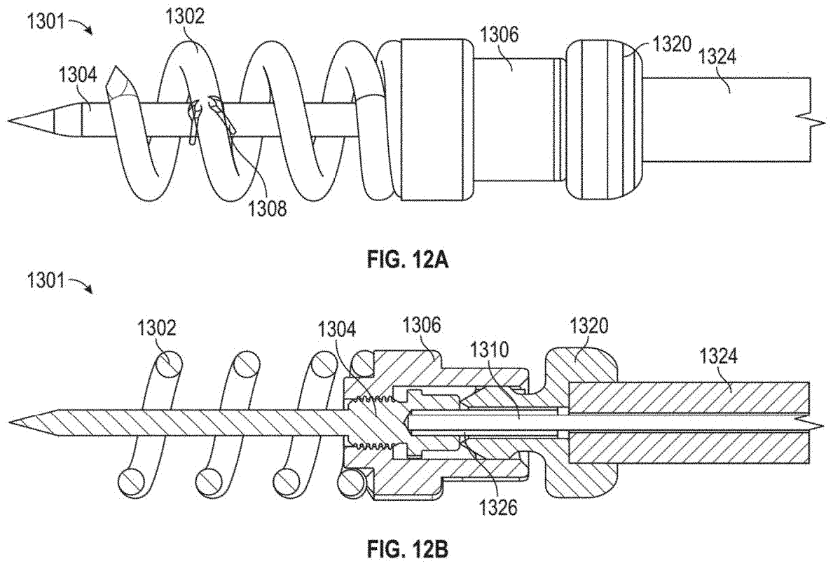

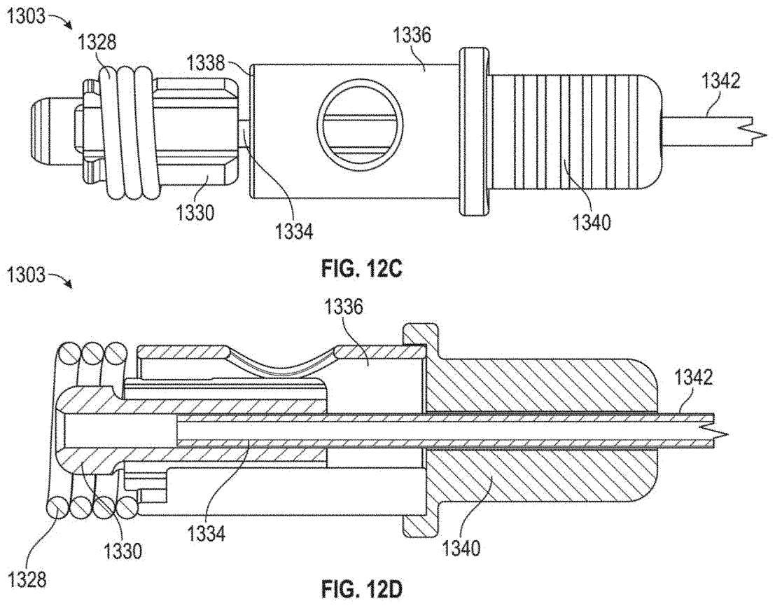

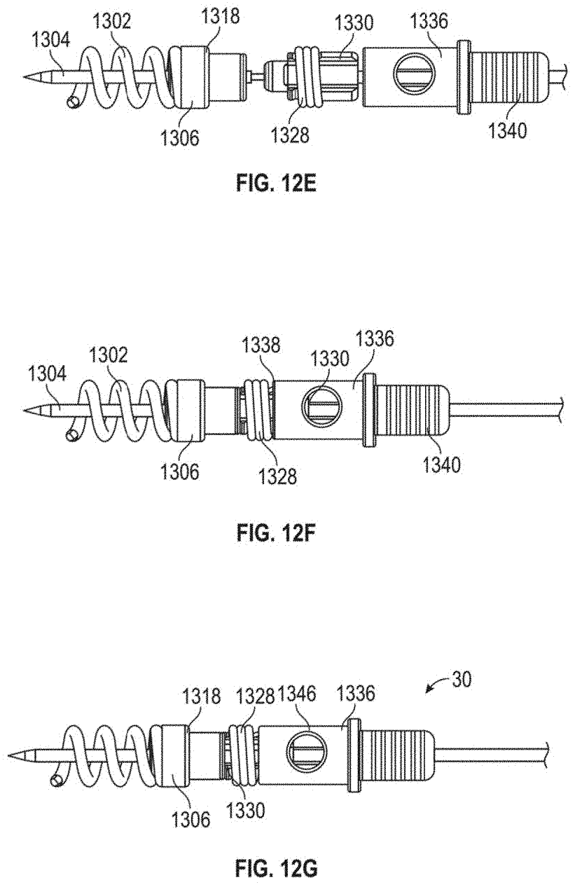

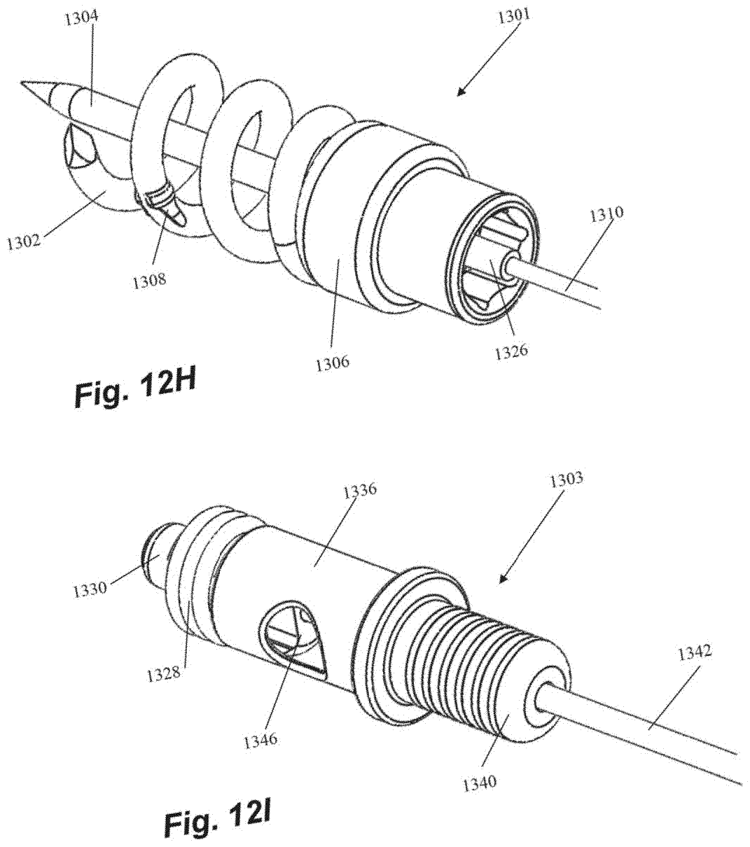

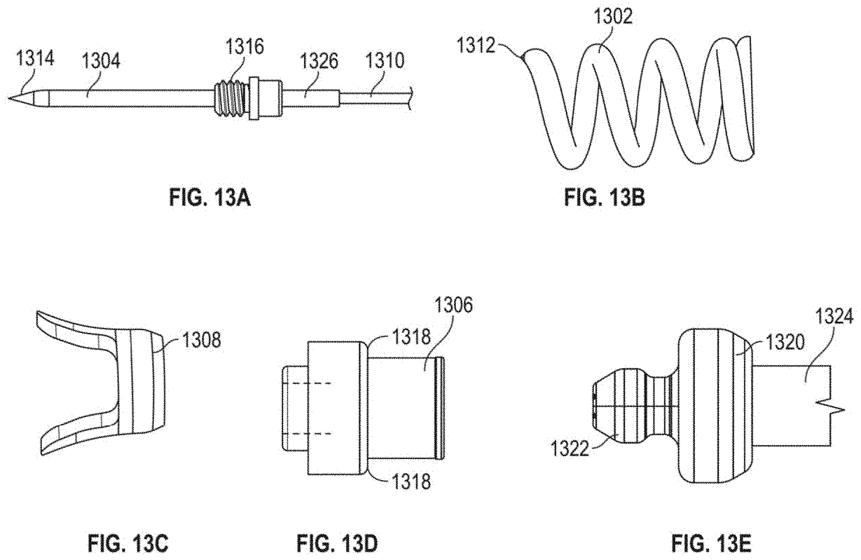

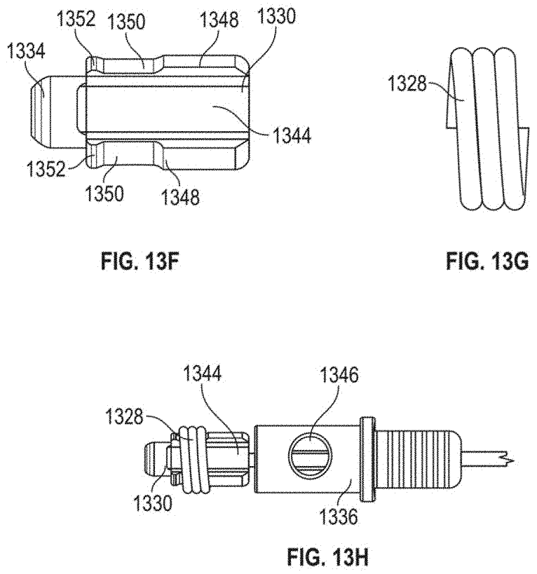

FIGS. 12A-121 depict various views of an anchor assembly that can be used with the methods as disclosed herein and FIGS. 13A-13H depict the various components thereof. FIGS. 8A-8J depict on example embodiment of a method of using such an anchor, but any of the embodiments disclosed herein could be employed and/or adapted for use with such an anchor. Anchor assembly includes an anchor delivery assembly 1301 and suture lock assembly 1303. Once the anchor delivery assembly 1301 is used to embed the anchor in the heart wall, the anchor delivery assembly 1301 is withdrawn and the suture lock assembly 1303 is used to deliver and lock the sutures to the anchor.

Anchor delivery assembly 1301 includes an anchor coil 1302 with a central stabilization needle 1304 extending longitudinally through the anchor coil 1302. Stabilization needle 1304 provides stability against the ventricular wall during anchor deployment and also provides the attachment to the tether 1310 that extends out of the body and is used to rotate the anchor assembly. Needle 1304 includes a sharpened distal tip 1314 configured to penetrate the heart tissue and a threaded portion 1316 that releasably secures the needle 1304 within internal threads in the anchor hub 1306. Anchor coil 1302 connects to anchor hub 1306, such as, for example, by welding, and can include an anti-backout feature. Anti-backout feature can be configured as a barb 1308 positioned around coil 1302 that keeps the coil 1302 from rotating back out of the tissue due to the natural rhythm of the heart. In embodiments, barb 1308 can be welded onto the coil 1302. Coil 1302 includes a sharpened distal tip 1312 configured to penetrate the tissue in the heart.

As noted above, anchor hub 1306 includes internal threading in a distal portion of anchor hub to releasably secure needle 1304 therein. Anchor hub 1306 also provides a proximally facing suture clamping surface 1318 extending around anchor hub 1306. Anchor driver 1320 includes a drive end 1322 that mates with corresponding internal geometry in the proximal portion of anchor hub 1306 to enable rotation of anchor hub 1306 with anchor driver 1320. Anchor driver 1320 can further includes a helical hollow strand (HHS) 1324 that extends out of the body and is twisted to provide the torque necessary to drive the anchor coil 1302 into the tissue. As can be seen in FIG. 8B, tether 1310 extends through anchor driver HHS 1324 and anchor driver 1320 to a connection within anchor hub 1306 to an aperture in the proximal end of stabilizing needle 1304. A stiffening tube 1326 can be threaded over tether 1310 within anchor hub 1306 to stiffen a small portion of the tether 1310 to provide better alignment to component that need to mate within the anchor hub 1306.

Suture lock assembly 1303 includes a suture lock configured as a spring 1328 that locks the suture by compressing the suture against the suture capture surface 1318 of the anchor hub 1306. Suture lock spring 1328 can be delivered to the anchor on a spring carrier 1330. Spring carrier 1330 can include a pair of upwardly raised ledges 1348 defining a suture channel 1344 therebetween. Each ledge 1348 can include a lock depression 1350 in which suture lock spring 1328 is seated for delivery and a retention lip 1352 projecting upwardly from lock depression 1350 to prevent inadvertent dislodgement of suture lock spring 1328. Spring carrier 1330 includes a distal portion 1332 that mates with the anchor hub 1306 to provide a tensioning point that is near the final point of suture lock to ensure proper tension is maintained. Tubing 1334 extends from spring carrier 1330 back out of the body to provide a hollow pathway for the tether 1310 to enable advancement of the spring carrier 1330 guided to the anchor hub 1306. In embodiments, tubing 1334 can be comprised of PEEK and can be bonded to the spring carrier. A pusher 1336 can be advanced over tubing 1334 and spring carrier 1330 and includes a distal surface 1338 configured to engage the suture spring lock 1328 to push the suture lock 1328 over the retention lips 1352 and off of the spring carrier 1330, onto the anchor hub 1306 and against the suture clamping surface 1318 of the anchor hub 1306. A pusher connector 1340 can be employed to connect the pusher to a catheter 1342 used to move the suture lock assembly 1303.

The routing of a suture 30 through suture lock assembly 1303 can be seen with respect to FIG. 8G. Outside of the body the suture 30 extending from the leaflet is threaded through the suture channel 1344 of the spring carrier 1330 beneath the suture lock spring 1328, into the pusher 1336 and out a suture aperture 1346 in the pusher. The suture 30 can then extend back through the anchor catheter out of the body for suture tensioning. When the suture lock spring 1328 is deployed with the pusher 1336, the suture 30 is crimped under tension between the suture lock spring 1328 and the suture capture surface 1318 of the anchor base 1306.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

1. A method of repairing a heart valve in a beating heart of a patient, comprising:
intravascularly accessing an interior of the heart;
inserting one or more sutures into a heart valve leaflet of the heart;
attaching the one or more sutures to a suture anchor exterior to the heart;
advancing the suture anchor into the heart and anchoring the suture anchor into a heart wall of the heart with an anchor delivery catheter;
adjusting a tension of the one or more sutures to achieve proper heart valve function; and
actuating a suture lock on the suture anchor to retain the one or more sutures at the suture anchor at the tension that achieves proper heart valve function.

2. The method of claim 1, wherein actuating the suture lock to retain the one or more sutures at the suture anchor includes rotating the suture lock to clamp the one or more sutures on the suture anchor.

3. The method of claim 1, wherein actuating the suture lock to retain the one or more sutures at the suture anchor includes longitudinally pushing the suture lock to clamp the one or more sutures on the suture anchor.

4. The method of claim 1, further comprising delivering the suture lock to the suture anchor in the heart.

5. The method of claim 1, wherein the one or more sutures are inserted into the heart valve leaflet before the suture anchor is anchored into the heart wall.

6. The method of claim 1, wherein the suture anchor is anchored into the heart wall before the one or more sutures are inserted into the heart valve.

7. The method of claim 6, further comprising positioning a tether to extend from the suture anchor in the heart wall out of the body.

8. The method of claim 6, wherein advancing the suture anchor into the heart and anchoring the suture into the heart wall includes anchoring an anchor body into the heart wall and attaching the one or more sutures to the suture anchor exterior to the heart includes attaching the one or more sutures to an anchor head, and further comprising advancing the anchor head from outside the body to the anchor body and attaching the anchor head to the anchor body.

9. The method of claim 1, wherein anchoring the suture anchor into the heart wall includes inserting a stabilizing needle into the heart wall and rotating the anchor to embed the anchor into the heart wall with the stabilizing needle holding a position of the anchor adjacent the heart wall as the anchor is initially rotated.

10. The method of claim 1, further comprising unlocking the one or more sutures on the suture anchor, readjusting the tension of the one or more sutures and re-actuating the suture lock to retain the sutures at the adjusted tension.

11. A system for repairing a heart valve in a beating heart of a patient, comprising:
an elongate flexible guide catheter configured to be inserted into the heart through the vasculature of the patient to provide a pathway into the heart from outside the body;
an elongate flexible anchor catheter configured to be inserted into the heart through the elongate flexible guide catheter;
a suture anchor configured to interface with a suture and be anchored in a heart wall of the heart with the anchor catheter to enable the suture to function as an artificial chordae extending between the anchor and a heart valve leaflet in the heart; and
a suture lock configured to selectively lock the suture on the suture anchor under tension.

12. The system of claim 11, wherein the suture lock is configured to selectively lock the suture on the suture anchor by being rotated to clamp the suture between the suture lock and the suture anchor.

13. The system of claim 12, wherein rotation of the suture anchor in an opposite direction releases the suture to enable a tension of the suture to be adjusted.

14. The system of claim 11, wherein the suture lock is configured to selectively lock the suture on the suture anchor by longitudinally pushing of the suture lock to clamp the suture between the suture lock and the suture anchor.

15. The system of claim 14, wherein longitudinally pulling of the suture lock releases the suture to enable a tension of the suture to be adjusted.

16. The system of claim 11, wherein the suture lock is configured to be delivered into the heart with the anchor catheter separately from the suture anchor.

17. The system of claim 11, further comprising a tether configured to be inserted into the heart with the suture anchor and configured to extend from the suture anchor out of the body.

18. The system of claim 17, wherein the suture anchor is configured to be delivered to the suture anchor along the tether.

19. The system of claim 11, wherein the suture anchor is configured to be rotated to embed the suture anchor into the heart wall.

20. The system of claim 19, wherein the suture anchor further comprises a stabilizing needle configured to be inserted into the heart wall to hold a position of the suture anchor adjacent the heart wall as the suture anchor is initially rotated
